(19) United States
(12) Patent Application Publication
(10) Pub. No.: US 2026/0193414 A1
(43) Pub. Date: Jul. 9, 2026

(54) SUPER ABSORBENT POLYMER MATERIALS

(71) Applicant: Planet Smart Ltd, London (GB)

(72) Inventor: Gerald Perry Marin, London (GB)

(21) Appl. No.: 19/445,069

(22) Filed: Jan. 9, 2026

Related U.S. Application Data
(63) Continuation of application No. PCT/IB25/61102, filed on Oct. 30, 2025.
(60) Provisional application No. 63/714,630, filed on Oct. 31, 2024.

(51) Int. Cl. C08G 73/10; A61F 13/15; B01J 20/26

(52) U.S. Cl. CPC C08G 73/1092; A61F 13/15252; B01J 20/267; A61F 2013/15146; A61F 2013/15463; B01J 2220/4812; B01J 2220/68; C08G 2210/00; C08G 2230/00

(57) ABSTRACT

Provided herein are biodegradable, superabsorbent polymers (SAPs) and hydrogels, methods of preparing the same, and articles of manufacture comprising the superabsorbent polymer or hydrogels. The superabsorbent polymer and hydrogels comprise a poly(aspartic acid) polymer crosslinked with one or more crosslinking agents and can be used to prepare articles of manufacture, such as diapers, sanitary pads, incontinence products, bandages, wound dressings, surgical pads, pet pads, bed pads, meat pads, and agricultural products.

### CROSS REFERENCE TO RELATED APPLICATIONS

This application is a continuation of PCT/IB2025/061102, filed on 30 Oct. 2025, which claims priority to U.S. Provisional Application No. 63/714,630, filed on 31 Oct. 2024, the contents of both of which are incorporated by reference in their entireties.

### BACKGROUND

Superabsorbent polymers (SAPs) have been instrumental in the evolution of articles having improved absorbency for aqueous fluids. While fossil-based polyacrylate SAPs have been the standard way to synthesize SAPs due to their high performance, there has recently been a move towards more sustainable biodegradable SAPs. However, bio-based and/or biodegradable polymer alternatives tend to suffer several drawbacks compared to industrially produced SAPs. For example, biodegradable SAPs may be very expensive to produce, depending on the types of materials and/or processes employed; exhibit functionality or performance issues, such as poor fluid retention, inadequate absorption underload, undesired colors and odors, and exuding off a slimy residue when they are over-saturated with fluids. There is limited availability of SAPs that are able to achieve industry-level performance levels to maintain maximum absorption while also keeping structural integrity under compression as well as ensuring biodegradability. Accordingly, there remains a need for improved biodegradable SAPs.

### SUMMARY

Provided herein are superabsorbent polymers (SAPs), comprising a poly(aspartic acid) polymer crosslinked with 1,8-diaminooctane; wherein the superabsorbent polymer has a water absorption capacity of from 10 g/g to 600 g/g; wherein the superabsorbent polymer has a saline absorption of from 10 g/g to 100 g/g; wherein the superabsorbent polymer has an absorption under load (AUL) of from 1 g/g to 100 g/g under a load of 0.7 psi; and wherein the superabsorbent polymer is degraded upon hydrolysis at a temperature of 15° C. or higher in 20 days when measured by ISO 20200:2024 or a comparable assay.

Also provided herein are SAPs, comprising a poly(aspartic acid) polymer crosslinked with 1,8-diaminooctane and cystamine; wherein the superabsorbent polymer has a water absorption capacity of from 10 g/g to 600 g/g, and wherein the superabsorbent polymer is degraded upon hydrolysis at a temperature of 15° C. in 20 days when measured by ISO 20200:2024 or a comparable assay. Also provided herein are SAPs, wherein the superabsorbent polymer has a water absorption capacity of from 20 g/g to 550 g/g. Also provided herein are SAPs, wherein the superabsorbent polymer has a water absorption capacity of from 30 g/g to 500 g/g. Also provided herein are SAPs, comprising a saline absorption of from than 20 g/g to 90 g/g. Also provided herein are SAPs, comprising a saline absorption of from 30 g/g to 80 g/g. Also provided herein are SAPs, comprising a saline absorption of from 40 g/g to 70 g/g. Also provided herein are SAPs, comprising an AUL of from 10 g/g to 90 g/g under a load of 0.7 psi. Also provided herein are SAPs, comprising an AUL of from 20 g/g to 80 g/g under a load of 0.7 psi. Also provided herein are SAPs, comprising a free swell capacity (FSC) of from 20 g/g to 100 g/g under a 0.9% saline solution. Also provided herein are SAPs, comprising a FSC of from 30 g/g to 90 g/g under a 0.9% saline solution. Also provided herein are SAPs, comprising a FSC of from 40 g/g to 80 g/g under a 0.9% saline solution. Also provided herein are SAPs, comprising a centrifuge retention capacity (CRC) of from 10 g/g to 50 g/g. Also provided herein are SAPs, comprising a CRC of from 20 g/g to 45 g/g. Also provided herein are SAPs, comprising a CRC of from 25 g/g to 40 g/g. Also provided herein are SAPs, wherein the superabsorbent polymer comprises 50% or more biobased carbons. Also provided herein are SAPs, wherein the superabsorbent polymer comprises 65% or more biobased carbons. Also provided herein are SAPs, wherein the superabsorbent polymer comprises 80% or more biobased carbons. Also provided herein are SAPs, wherein the 1,8-diaminooctane is present at a concentration of from 0.1 wt % to 15 wt % of the superabsorbent polymer. Also provided herein are SAPs, wherein the 1,8-diaminooctane is present at a concentration of from 1 wt % to 10 wt % of the superabsorbent polymer. Also provided herein are SAPs, wherein the superabsorbent polymer has a whiteness index of L* in a range of from 95 to 100, a* in a range of from −1.5 to −1.1 and b* in a range of from 4.2 to 4.7. Also provided herein are SAPs further comprising a surface crosslinker selected from an epoxy; ethylene glycol diglycidyl ether (EGDGE); poly(ethylene glycol) diglycidyl ether (PEGDGE); ethylene glycol; diethyleneglycol diglycidyl ether; 1,4-butanediol diglycidyl ether; glycerol polyglycidyl ether; sorbitol polyglycidyl ether; 2-oxazolidinone; N-(2-hydroxy ethyl)-2 oxazolidinone (HEONON); ethylene carbonate; 1,4 butane diol; glycerol; ethylene diamine; and 1,4 butane diamine. Also provided herein are SAPs, wherein the 1,8-diaminooctane is present at a concentration of from 2 wt % to 5 wt % of the superabsorbent polymer. Also provided herein are SAPs, wherein the 1,8-diaminooctane is present at a concentration of 2.5 wt % of the superabsorbent polymer. Also provided herein are SAPs, wherein the cystamine is present at a concentration of from 0.1 wt % to 10 wt % of the superabsorbent polymer. Also provided herein are SAPs, wherein the cystamine is present at a concentration of from 1 wt % to 5 wt % of the superabsorbent polymer. Also provided herein are SAPs, wherein the cystamine is present at a concentration of from 2 wt % to 4 wt % of the superabsorbent polymer. Also provided herein are SAPs, wherein the cystamine is present at a concentration of 2.5 wt % of the superabsorbent polymer. Also provided herein are SAPs, wherein the superabsorbent polymer is hydrolyzed on exposure to an enzymatic hydrolysis or chemical hydrolysis. Also provided herein are SAPs, wherein the enzymatic hydrolysis occurs upon contact of the superabsorbent polymer with one or more enzymes or one or more microorganisms. Also provided herein are SAPs, wherein the one or more enzymes comprises one or more lipolytic enzymes, one or more proteolytic enzymes, one or more amylolytic enzymes, one or more cellulolytic enzymes, or any combination thereof. Also provided herein are SAPs, wherein the one or more lipolytic enzymes comprises one or more carboxylesterases and/or lipases. Also provided herein are SAPs, wherein the one or more proteolytic enzymes comprises one or more serine proteases, cysteine proteases, threonine proteases, aspartic proteases, metalloproteases, glutamic proteases, or any combination thereof. Also provided herein are SAPs, wherein the one or more serine proteases comprises one or more trypsins, chymotrypsins, thrombins, subtilisins, or any combination thereof. Also provided herein are SAPs, wherein the one or more amylolytic enzymes comprise one or more α-amylases, β-amylases, glucoamylases, or any combination thereof. Also provided herein are SAPs, wherein the one or more microorganisms comprises a bacterium or a yeast. Also provided herein are SAPs, wherein the one or more microorganisms comprises psychrophiles, mesophiles, thermophiles, actinomycetes, saprophytes, Absidia, Acremonium, Alternaria, amerospore, Arthrinium, ascospore, Aspergillus, Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus nidulans, Aspergillus ochraceus, Aspergillus oryzae, Aspergillus parasiticus, Aspergillus penicilloides, Aspergillus restrictus, Aspergillus sydowii, Aspergillus terreus, Aspergillus ustus, Aspergillus versicolor, Aspergillus/Penicillium-like, Aureobasidium, basidiomycetes, basidiospore, Bipolaris, Blastomyces, B. borstelensis, Botrytis, Candida, Cephalosporium, Chaetomium, Cladosporium, Cladosporium fulvum, Cladosporium herbarum, Cladosporium macrocarpum, Cladosporium sphaerospermum, conidia, conidium, conidobolus, Cryptococcus neoformans, cryptostroma corticale, Cunninghamella, Curvularia, dreschlera, Epicoccum, Epidermophyton, Fungus, Fusarium, Fusarium solani, Geotrichum, Gliocladium, Helicomyces, Helminthosporium, Histoplasma, humicula, Hyaline mycelia, Memnoniella, Microsporum, mold, Monilia, Mucor, mycelium, myxomycetes, Nigrospora, Oidium, Paecilomyces, papulospora, Penicillium, Periconia, perithecium, Peronospora, phaeohyphomycosis, Phoma, Pithomyces, RhizoMucor, Rhizopus, Rhodococcus, Rhodotorula, rusts, Saccharomyces, Scopulariopsis, Sepedonium, Serpula lacrymans, smuts, Spegazzinia, spore, Sporoschisma, Sporothrix, Sporotrichum, Stachybotrys, Stemphylium, Syncephalastrum, Thermononespore fusca DSM43793, Torula, Trichocladium, Trichoderma, Trichophyton, Trichothecium, tritirachium, Ulocladium, Verticillium, Wallemia, yeast, or any combination thereof. Also provided herein are SAPs, wherein the superabsorbent polymer is degradable upon exposure to solar radiation. Also provided herein are SAPs, wherein the solar radiation comprises gamma rays, UV rays, visible light, infrared rays, x-rays, microwave radiation, radio waves, or any combination thereof. Also provided herein are SAPs, wherein the superabsorbent polymer degrades upon contact with an effective amount of environmental moisture. Also provided herein are SAPs, wherein the environmental moisture comprises a body of water, soil moisture, waste moisture, or an atmospheric humidity. Also provided herein are SAPs, wherein environmental moisture comprises a relative humidity of 10% to 60% or a moisture content of from 10% to 65%, or greater. Also provided herein are SAPs, wherein 75% or greater of the superabsorbent polymer is degraded at a temperature of 15° C. in 20 days when measured by ISO 20200:2024 or a comparable assay. Also provided herein are SAPs, wherein 80% or greater of the superabsorbent polymer is degraded at a temperature of 15° C. in 20 days when measured by ISO 20200:2024 or a comparable assay. Also provided herein are SAPs, wherein 85% or greater of the superabsorbent polymer is degraded at a temperature of 15° C. in 20 days when measured by ISO 20200:2024 or a comparable assay. Also provided herein are SAPs, wherein 90% or greater of the superabsorbent polymer is degraded at a temperature of 15° C. in 20 days when measured by ISO 20200:2024 or a comparable assay. Also provided herein are SAPs, wherein 95% or greater of the superabsorbent polymer is degraded at a temperature of 15° C. in 20 days when measured by ISO 20200:2024 or a comparable assay. Also provided herein are SAPs, wherein 99% or greater of the superabsorbent polymer is degraded at a temperature of 25° C. in 20 days when measured by ISO 20200:2024 or a comparable assay. Also provided herein are SAPs, wherein 100% of the superabsorbent polymer is degraded at a temperature of 15° C. in 20 days when measured by ISO 20200:2024 or a comparable assay.

Provided herein are biodegradable poly(aspartic acid) hydrogels, comprising a superabsorbent biopolymer comprising a poly(aspartic acid) polymer crosslinked with 1,8-diaminooctane and/or cystamine; and one or more fluids, a charged species, a biological molecule, an organic compound, a metal, or combinations thereof; wherein the superabsorbent polymer has a water absorption capacity of from 10 g/g to 600 g/g, and wherein the biodegradable poly(aspartic acid) hydrogel is degraded upon hydrolysis at a temperature of 15° C. in 20 days when measured by ISO 20200:2024 or a comparable assay. Also provided herein are biodegradable poly(aspartic acid) hydrogels, wherein the superabsorbent polymer binds, adsorbs, absorbs, dissolves, sequesters, chelates, or any combination thereof the one or more fluids, the charged species, the biological molecule, the organic compound, the metal, or any combination thereof. Also provided herein are biodegradable poly(aspartic acid) hydrogels, wherein the one or more fluids comprises water, hydrocarbon, oil, alcohol, an aqueous solution, a non-aqueous solution, an ionic solution, a biological fluid, a gas, a waste-water, a fracking fluid, or any combination thereof. Also provided herein are biodegradable poly(aspartic acid) hydrogels, wherein the biological fluid comprises blood, urine, fecal water, vomit, or any combination thereof. Also provided herein are biodegradable poly(aspartic acid) hydrogels, wherein the biological molecule comprises a peptide, a polypeptide, a nucleic acid, an oligonucleotide, a carbohydrate, a lipid, or any combination thereof. Also provided herein are biodegradable poly(aspartic acid) hydrogels, wherein the charged species comprises potassium ions (K⁺), calcium ions (Ca²⁺), sodium ions (Na⁺), chloride ions (C¹⁻), fluoride ions (F⁻), phosphite ions (PO₃³⁻), sulfate ions (SO₄²⁻), sulfite ions (SO₃²⁻), phosphate ions (PO₄³⁻), polyatomic ions, metal ions, charged biological molecules, or any combination thereof. Also provided herein are biodegradable poly(aspartic acid) hydrogels, wherein the metal comprises lead, mercury, cadmium, arsenic, copper, chromium, thallium, selenium, zinc, calcium, magnesium, silver, boron, lithium, ammonium, barium, strontium, manganese, silver, cesium, zinc, cadmium, selenium, calcium, magnesium, iron, radium, mercury, copper, lead, nickel, chromium, arsenic, gold, uranium, a carbonate of any of the foregoing, a sulphate of any of the foregoing, and/or phosphate of any of the foregoing, or any combination thereof. Provided herein are article of manufactures comprising a superabsorbent polymer comprising a poly(aspartic acid) polymer crosslinked with 1,8-diaminooctane and cystamine, (a) wherein the superabsorbent polymer has a water absorption capacity of from 10 g/g to 600 g/g, (b) wherein the superabsorbent polymer is degraded upon hydrolysis at a temperature of 15° C. in 20 days when measured by ISO 20200:2024 or a comparable assay; and wherein the article of manufacture is a diaper, a sanitary pad, an incontinence product, a bandage, a wound dressing, a surgical pad, a pet pad, a bed pad, or a meat pad.

Also provided herein are SAPs, comprising a crosslinked poly(aspartic acid) polymer; wherein the superabsorbent polymer has: a whiteness index of L* in a range of from 97 to 100, a* in a range of from −1.4 to −1.2 and b* in a range of from 4.3 to 4.6; a water absorption capacity of at least 500 g/g; and a free swell capacity (FSC) in a range of from 30 g/g to 80 g/g. Also provided herein are SAPs, wherein the SAP has a whiteness index with L* in a range of from 99 to 100, a* in a range of from −1.4 to −1.3 and b* in a range of from 4.4 to 4.5 Also provided herein are SAPs, wherein the SAP has a whiteness index of L*=95.5, a*=1.2 and b*=4.5. Also provided herein are SAPs, wherein the SAP has a water absorption capacity of from 900 g/g to 1400 g/g. Also provided herein are SAPs, wherein the SAP has a water absorption capacity of from 1000 g/g to 1200 g/g. Also provided herein are SAPs, comprising a free swell capacity (FSC) of from 40 g/g to 80 g/g under a 0.9% saline solution. Also provided herein are SAPs, comprising a FSC of from 50 g/g to 75 g/g under a 0.9% saline solution. Also provided herein are SAPs, comprising a centrifuge retention capacity (CRC) of from 30 g/g to 70 g/g. Also provided herein are SAPs, comprising a CRC of from 40 g/g to 70 g/g. Also provided herein are SAPs, comprising a CRC of from 60 g/g to 70 g/g. Also provided herein are SAPs, comprising an absorbance under load (AUL) of from 8 g/g to 30 g/g under a load of 0.7 psi. Also provided herein are SAPs, comprising an AUL of from 10 g/g to 20 g/g under a load of 0.7 psi. Also provided herein are SAPs, further comprising a surface crosslinker. Also provided herein are SAPs, wherein the surface crosslinker is an epoxy; ethylene glycol diglycidyl ether (EGDGE); poly(ethylene glycol) diglycidyl ether (PEGDGE); ethylene glycol; diethyleneglycol diglycidyl ether; 1,4-butanediol diglycidyl ether; glycerol polyglycidyl ether; sorbitol polyglycidyl ether; 2-oxazolidinone; N-(2-hydroxy ethyl)-2 oxazolidinone (HEONON); ethylene carbonate; 1,4 butane diol; glycerol; ethylene diamine; or 1, 4 butane diamine. Also provided herein are SAPs, wherein the surface crosslinker is EGDGE or PEGDGE.

Provided herein are methods of preparing a superabsorbent polymer comprising the steps: (a) polymerizing aspartic acid monomers using a phosphoric acid (H₃PO₄) catalyst to form a polysuccinimide polymer; (b) crosslinking the polysuccinimide polymer in an organic solvent using one or more crosslinking agents to form a crosslinked polysuccinimide polymer; and (c) hydrolyzing the crosslinked polysuccinimide polymer using sodium carbonate (Na₂CO₃) to form a sodium salt form of crosslinked poly(aspartic acid) polymer. Also provided herein are method of preparing a superabsorbent polymer, wherein the polymerizing of the aspartic acid monomers using the phosphoric acid (H₃PO₄) catalyst is performed at a temperature of from 150° C. to 300° C. Also provided herein are method of preparing a superabsorbent polymer, wherein the polymerizing of the aspartic acid monomers using the phosphoric acid (H₃PO₄) catalyst is performed at a temperature of from 150° C. to 250° C. Also provided herein are method of preparing a superabsorbent polymer, wherein the polymerizing of the aspartic acid monomers using the phosphoric acid (H₃PO₄) catalyst is performed at a temperature of from 150° C. to 200° C. Also provided herein are method of preparing a superabsorbent polymer, wherein the one or more crosslinking agents comprises 1,8-diaminooctane. Also provided herein are method of preparing a superabsorbent polymer, wherein the 1,8-diaminooctane is present in a concentration of from 0.1 wt % to 10 wt % of the superabsorbent polymer. Also provided herein are method of preparing a superabsorbent polymer, wherein the 1,8-diaminooctane is present in a concentration of from 1 wt % to 5 wt % of the superabsorbent polymer. Also provided herein are method of preparing a superabsorbent polymer, wherein the 1,8-diaminooctane is present in a concentration of from 2 wt % to 4 wt % of the superabsorbent polymer. Also provided herein are methods of preparing a superabsorbent polymer, wherein the 1,8-diaminooctane is present in a concentration of 2.5 wt % of the superabsorbent polymer. Also provided herein are methods of preparing a superabsorbent polymer, wherein the one or more crosslinking agents comprises cystamine. Also provided herein are methods of preparing a superabsorbent polymer, wherein the cystamine is present in a concentration of from 0.1 wt % to 10 wt % of the superabsorbent polymer. Also provided herein are methods of preparing a superabsorbent polymer, wherein the cystamine is present in a concentration of from 1 wt % to 5 wt % of the superabsorbent polymer. Also provided herein are methods of preparing a superabsorbent polymer, wherein the cystamine is present in a concentration of from 2 wt % to 4 wt % of the superabsorbent polymer. Also provided herein are methods of preparing a superabsorbent polymer, wherein the cystamine is present in a concentration of 2.5 wt % of the superabsorbent polymer. Also provided herein are methods of preparing a superabsorbent polymer, wherein the one or more crosslinking agents comprise 1,8-diaminooctane and cystamine. Also provided herein are methods of preparing a superabsorbent polymer, wherein the 1,8-diaminooctane is present in a concentration of 2.5 wt % of the superabsorbent polymer and cystamine is present in a concentration of 2.5 wt % of the superabsorbent polymer.

Provided herein are methods of preparing a superabsorbent polymer comprising the steps: (a) polymerizing aspartic acid monomers using a phosphoric acid (H₃PO₄) catalyst to form a polysuccinimide polymer; (b) crosslinking the polysuccinimide polymer in an organic solvent using 1,8-diaminooctane as a crosslinking agent to form a crosslinked polysuccinimide polymer; and (c) hydrolyzing the crosslinked polysuccinimide polymer using sodium carbonate (Na₂CO₃) to form a sodium salt of crosslinked poly(aspartic acid) polymer.

Provided herein are methods of preparing a superabsorbent polymer comprising the steps: (a) polymerizing aspartic acid monomers using a phosphoric acid (H₃PO₄) catalyst to form a polysuccinimide polymer; (b) crosslinking the polysuccinimide polymer in an organic solvent using 1,8-diaminooctane and cystamine as crosslinking agents to form a crosslinked polysuccinimide polymer; and (c) hydrolyzing the crosslinked polysuccinimide polymer using sodium carbonate (Na₂CO₃) to form a sodium salt of crosslinked poly(aspartic acid) polymer. Also provided herein are methods, wherein the organic solvent comprises dimethylsulfoxide (DMSO). Also provided herein are methods, wherein the organic solvent comprises diphenyl acetone (DPA). Also provided herein are methods, wherein the organic solvent comprises cyrene. Also provided herein are methods, wherein the superabsorbent polymer binds, adsorbs, absorbs, dissolves, sequesters, chelates, or any combination thereof, one or more fluids, a charged species, a biological molecule, an organic compound, or metal, or any combination thereof by at least 5%, to at least 200% or greater when compared to a super absorbent polymer prepared using a conventional method.

Provided herein are superabsorbent polymer intermediates comprising: (a) a polysuccinimide polymer; (b) a poly(aspartic acid) polymer or salt thereof, crosslinked with one or more crosslinking agents; (c) a phosphoric acid (H₃PO₄) catalyst; (d) an organic solvent; (e) sodium carbonate (Na₂CO₃); and (f) a salt of crosslinked poly(aspartic acid) polymer. Also provided herein are superabsorbent polymer intermediates, wherein the salt of the crosslinked poly(aspartic acid) polymer is a sodium salt or a potassium salt. Also provided herein are superabsorbent polymer intermediates, wherein the one or more crosslinking agents comprises 1,8-diaminooctane. Also provided herein are superabsorbent polymer intermediates, wherein the 1,8-diaminooctane is present at a concentration of from 0.1 wt % to 10 wt % of the superabsorbent polymer. Also provided herein are superabsorbent polymer intermediates, wherein the 1,8-diaminooctane is present at a concentration of from 1 wt % to 5 wt % of the superabsorbent polymer. Also provided herein are superabsorbent polymer intermediates, wherein the 1,8-diaminooctane is present at a concentration of from 2 wt % to 4 wt % of the superabsorbent polymer. Also provided herein are superabsorbent polymer intermediates, wherein the 1,8-diaminooctane is present at a concentration of 2.5 wt % of the superabsorbent polymer. Also provided herein are superabsorbent polymer intermediates, wherein the one or more crosslinking agents comprises cystamine. Also provided herein are superabsorbent polymer intermediates, wherein the cystamine is present in a concentration of from 0.1 wt % to 10 wt % of the superabsorbent polymer. Also provided herein are superabsorbent polymer intermediates, wherein the cystamine is present in a concentration of from 1 wt % to 5 wt % of the superabsorbent polymer. Also provided herein are superabsorbent polymer intermediates, wherein the cystamine is present in a concentration of from 2 wt % to 4 wt % of the superabsorbent polymer. Also provided herein are superabsorbent polymer intermediates, wherein the cystamine is present in a concentration of 2.5 wt % of the superabsorbent polymer. Also provided herein are superabsorbent polymer intermediates, wherein the one or more crosslinking agents comprises 1,8-diaminooctane and cystamine. Also provided herein are superabsorbent polymer intermediates, wherein the 1,8-diaminooctane is present in a concentration of 2.5 wt % of the superabsorbent polymer and the cystamine is present in a concentration of 2.5 wt % of the superabsorbent polymer. Also provided herein are superabsorbent polymer intermediates, wherein the organic solvent comprises dimethylsulfoxide (DMSO). Also provided herein are superabsorbent polymer intermediates, wherein the organic solvent comprises diphenyl acetone (DPA). Also provided herein are superabsorbent polymer intermediates, wherein the organic solvent comprises cyrene.

Provided herein are systems of preparing a superabsorbent polymer comprising: (a) aspartic acid monomers; (b) a phosphoric acid (H₃PO₄) catalyst; (c) a crosslinked polysuccinimide polymer; (d) an organic solvent; (e) one or more crosslinking agents; and (f) sodium carbonate (Na₂CO₃) base. Also provided herein are systems, wherein the one or more crosslinking agents comprises 1,8-diaminooctane. Also provided herein are systems, wherein the 1,8-diaminooctane is present at a concentration of from 0.1 wt % to 10 wt % of the superabsorbent polymer. Also provided herein are systems, wherein the 1,8-diaminooctane is present at a concentration of from 1 wt % to 5 wt % of the superabsorbent polymer. Also provided herein are systems, wherein the 1,8-diaminooctane is present at a concentration of from 2 wt % to 4 wt % of the superabsorbent polymer. Also provided herein are systems, wherein the 1,8-diaminooctane is present at a concentration of 2.5 wt % of the superabsorbent polymer. Also provided herein are systems, wherein the one or more crosslinking agents comprises cystamine. Also provided herein are systems, wherein the cystamine is present in a concentration of from 0.1 wt % to 10 wt % of the superabsorbent polymer. Also provided herein are systems, wherein the cystamine is present in a concentration of from 1 wt % to 5 wt % of the superabsorbent polymer. Also provided herein are systems, wherein the cystamine is present in a concentration of from 2 wt % to 4 wt % of the superabsorbent polymer. Also provided herein are systems, wherein the cystamine is present in a concentration of 2.5 wt % of the superabsorbent polymer. Also provided herein are systems, wherein the one or more crosslinking agents comprises 1,8-diaminooctane and cystamine. Also provided herein are systems, wherein the 1,8-diaminooctane is present in a concentration of 2.5 wt % of the superabsorbent polymer and the cystamine is present in a concentration of 2.5 wt % of the superabsorbent polymer. Also provided herein are systems, wherein the one or more crosslinking agents comprise an amine based crosslinker. Also provided herein are systems, wherein the one or more crosslinking agents comprise ethylenediamine; 1,4-butanediamine; 1,5-pentanediamine; 1,6-hexamethylenediamine; 1, 10-diaminodecane; 1,12-diaminododecane; L-lysine; L-ornithine; diethylenetriamine; triethylenetetramine; 1,4-diaminocyclohexane; p-phenylenediamine, or tris(2-aminoethyl)amine. Also provided herein are systems, wherein the one or more crosslinking agents comprise epoxide based crosslinkers. Also provided herein are systems, wherein the one or more crosslinking agents comprise ethylene glycol diglycidyl ether (EGDGE); poly(ethylene glycol) diglycidyl ether (PEGDGE); glycerol polyglycidyl ether, or sorbitol polyglycidyl ether.

Also provided herein are systems, wherein the organic solvent comprises dimethylsulfoxide (DMSO). Also provided herein are systems, wherein the organic solvent comprises diphenyl acetone (DPA). Also provided herein are systems, wherein the organic solvent comprises cyrene.

Provided herein are articles of manufacture comprising a superabsorbent polymer comprising a poly(aspartic acid) polymer crosslinked with 1,8-diaminooctane; wherein the superabsorbent polymer has a water absorption capacity of from 10 g/g to 600 g/g; wherein the superabsorbent polymer has a saline absorption of from 10 g/g to 100 g/g; wherein the superabsorbent polymer has an absorption under load (AUL) of from 1 g/g to 100 g/g under a load of 0.7 psi; and wherein the superabsorbent polymer is degraded upon hydrolysis at a temperature of 15° C. in 20 days when measured by ISO 20200:2024 or a comparable assay, and wherein the article of manufacture is a diaper, a sanitary pad, an incontinence product, a bandage, a wound dressing, a surgical pad, a pet pad, a bed pad, or a meat pad.

Provided herein are articles of manufacture comprising a superabsorbent polymer comprising a poly(aspartic acid) polymer crosslinked with 1,8-diaminooctane and cystamine, wherein the superabsorbent polymer has a water absorption capacity of from 10 g/g to 600 g/g, wherein the superabsorbent polymer is degraded upon hydrolysis at a temperature of 15° C. in 20 days when measured by ISO 20200:2024 or a comparable assay; and wherein the article of manufacture is a diaper, a sanitary pad, an incontinence product, a bandage, a wound dressing, a surgical pad, a pet pad, a bed pad, or a meat pad. Also provided herein are articles of manufacture, wherein the 1,8-diaminooctane is present at a concentration of from 0.1 wt % to 10 wt % of the superabsorbent polymer. Also provided herein are articles of manufacture, wherein the 1,8-diaminooctane is present at a concentration of from 1 wt % to 5 wt % of the superabsorbent polymer. Also provided herein are articles of manufacture, wherein the 1,8-diaminooctane is present at a concentration of from 2 wt % to 4 wt % of the superabsorbent polymer. Also provided herein are articles of manufacture, wherein the 1,8-diaminooctane is present at a concentration of 2.5 wt % of the superabsorbent polymer. Also provided herein are articles of manufacture, wherein the cystamine is present in a concentration of from 0.1 wt % to 10 wt % of the superabsorbent polymer. Also provided herein are articles of manufacture, wherein the cystamine is present in a concentration of from 1 wt % to 5 wt % of the superabsorbent polymer. Also provided herein are articles of manufacture, wherein the cystamine is present in a concentration of from 2 wt % to 4 wt % of the superabsorbent polymer. Also provided herein are articles of manufacture, wherein the cystamine is present in a concentration of 2.5 wt % of the superabsorbent polymer.

Also provided herein are biodegradable poly(aspartic acid) (PASP) hydrogels, comprising a superabsorbent biopolymer comprising a poly(aspartic acid) polymer crosslinked with 1,8-diaminooctane and/or cystamine; wherein the poly(aspartic acid) biopolymer comprises particles that are surface crosslinked with ethylene glycol diglycidyl ether (EGDGE), poly(ethylene glycol) diglycidyl ether (PEGDGE), or ethylene glycol and wherein the superabsorbent biopolymer has a whiteness index of L* in a range of from 97 to 100, a* in a range of from −1.4 to −1.2 and b* in a range of from 4.3 to 4.6. Also provided herein are biodegradable PASP hydrogels, wherein the superabsorbent biopolymer has a water absorption capacity of from 700 g/g to 1400 g/g. Also provided herein are biodegradable PASP hydrogels, comprising a free swell capacity (FSC) of from 30 g/g to 80 g/g under a 0.9% saline solution. Also provided herein are biodegradable PASP hydrogels, comprising a centrifuge retention capacity (CRC) of from 30 g/g to 70 g/g. Also provided herein are biodegradable PASP hydrogels, comprising an absorbance under load (AUL) of from 8 g/g to 20 g/g under a load of 0.7 psi.

Also provided herein are methods of preparing SAPs comprising the steps: (a) polymerizing aspartic acid monomers using an acid catalyst to form a polysuccinimide polymer; (b) crosslinking the polysuccinimide polymer in an organic solvent using one or more crosslinking agents to form a crosslinked polysuccinimide polymer; (c) hydrolyzing the crosslinked polysuccinimide polymer using base to form a salt form of crosslinked poly(aspartic acid) polymer; and (d) surface crosslinking the poly(aspartic acid) polymer with a surface crosslinker comprising ethylene glycol diglycidyl ether (EGDGE) or poly(ethylene glycol) diglycidyl ether (PEGDGE), or ethylene glycol, wherein the superabsorbent polymer a whiteness index of L* in a range of from 97 to 100, a* in a range of from −1.4 to −1.2 and b* in a range of from 4.3 to 4.6. Also provided herein are methods of preparing SAPs, wherein the acid catalyst is a phosphoric acid (H₃PO₄) catalyst. Also provided herein are methods of preparing SAPs, wherein the base is sodium carbonate (Na₂CO₃) and the salt is a sodium salt.

Also provided herein are articles of manufacture comprising SAPs comprising a poly(aspartic acid) polymer crosslinked with 1,8-diaminooctane; wherein the superabsorbent polymer has a whiteness index of L* in a range of from 97 to 100, a* in a range of from −1.4 to −1.2 and b* in a range of from 4.3 to 4.6; a water absorption capacity in a range of from 700 g/g to 1400 g/g; and a free swell capacity (FSC) in a range of from 30 g/g to 80 g/g., and wherein the article of manufacture is a diaper, a sanitary pad, an incontinence product, a bandage, a wound dressing, a surgical pad, a pet pad, a bed pad, or a meat pad. Also provided herein are articles of manufacture comprising SAPs, wherein the article of manufacture is substantially free of microplastics. Also provided herein are articles of manufacture comprising SAPs, wherein the superabsorbent polymer comprises less than 5% monomers.

Also provided herein are biodegradable PASP hydrogels, comprising a SAP, comprising a poly(aspartic acid) polymer crosslinked with 1,8-diaminooctane; wherein the superabsorbent polymer has a water absorption capacity of from 10 g/g to 600 g/g; wherein the superabsorbent polymer has a saline absorption of from 10 g/g to 100 g/g; wherein the superabsorbent polymer has an absorption under load (AUL) of from 1 g/g to 100 g/g under a load of 0.7 psi; and wherein the biodegradable poly(aspartic acid) hydrogel is degraded upon hydrolysis at a temperature of 15° C. in 20 days when measured by ISO 20200:2024 or a comparable assay.

Also provided herein are biodegradable PASP hydrogels, comprising a SAP, comprising a poly(aspartic acid) polymer crosslinked with 1,8-diaminooctane and cystamine; wherein the superabsorbent polymer has a water absorption capacity of from 10 g/g to 600 g/g, and wherein the biodegradable poly(aspartic acid) hydrogel is degraded upon hydrolysis at a temperature of 15° C. in 20 days when measured by ISO 20200:2024 or a comparable assay.

Also provided herein are articles of manufacture comprising said biodegradable PASP hydrogel disclosed herein. Also provided herein are articles of manufacture comprising said biodegradable PASP hydrogel, wherein said article of manufacture is selected from the group consisting of: (a) devices for controlled release of water, nutrients or phytopharmaceuticals in agriculture, (b) absorbent products for dewatering mine tailings and sludge, and (c) biomedical devices.

### BRIEF DESCRIPTION OF THE DRAWINGS

The novel features of the disclosure are set forth with particularity in the appended claims. An improved understanding of the novel features and advantages of the present disclosure will be obtained by reference to the following detailed description that sets forth illustrative embodiments, in which the principles of the disclosure are utilized, and the accompanying drawings of which:

FIG. 1 depicts an exemplary test superabsorbent polymer prior to being subjected to tests described herein.

FIG. 2 depicts the exemplary test superabsorbent polymer of FIG. 1 after mixing with water.

FIG. 3 depicts the exemplary test superabsorbent polymer of FIG. 1 after mixing with water and an exemplary enzyme.

FIG. 4 depicts the exemplary test superabsorbent polymer of FIG. 1 after mixing with saline and an exemplary enzyme.

FIG. 5 depicts the exemplary test superabsorbent polymer of FIG. 2 in a 50 μm mesh bag after 14 days.

FIG. 6 depicts the exemplary test superabsorbent polymer of FIG. 3 in a 50 μm mesh bag after 14 days.

FIG. 7 depicts the exemplary test superabsorbent polymer of FIG. 4 in a 50 μm mesh bag after 14 days.

FIG. 8 depicts compost growth in an exemplary test superabsorbent polymer after 14 days.

FIG. 9 depicts the exemplary test superabsorbent polymer of FIG. 2 after 20 days.

FIG. 10 depicts the exemplary test superabsorbent polymer of FIG. 3 after 20 days.

FIG. 11 depicts the exemplary test superabsorbent polymer of FIG. 4 after 20 days.

FIG. 12 depicts the comparative masses of the exemplary test SAPs of FIG. 2, FIG. 3, and FIG. 4, partially dried, at day 20.

FIG. 13 depicts a sample of acidified polyaspartic acid (PASP) coated with 1.5 wt % ethylene glycol diglycidyl ether (EGDGE) after testing in a 0.9 wt % saline solution.

### DETAILED DESCRIPTION

All publications, patents, and patent applications mentioned in this specification are herein incorporated by reference to the same extent as if each individual publication, patent, or patent application was specifically and individually indicated to be incorporated by reference.

### Definitions

Unless defined otherwise, all technical and scientific terms used herein have the same meaning as commonly understood by a person of ordinary skill in the art to which this disclosure belongs. The following definitions are intended to also include their various grammatical forms, where applicable.

As used herein, the singular form “a,” “an,” and “the” include plural references unless the context clearly dictates otherwise. For example, the term “a crosslinking agent” includes a plurality of crosslinking agents, including mixtures thereof.

As used herein, the term “about” when referring to a measurable value such as an amount or concentration and the like and, unless stated otherwise, is meant to encompass variations of +/−20%, which includes +/−10%, 5%, 1%, 0.5%, or even 0.1% of the specified amount.

As used herein, the term “Absorption under load” or “AUL” refers to the permeability dependent absorption under pressure of saline solution by gravimetric measurement as determined by EDANA NWSP 242.0.R2 or a comparable assay. The test is performed under a 0.9% saline solution.

As used herein, the term “biobased carbon” refers to carbons derived from or synthesized by a biological organism and can be considered a renewable resource since it can be generated by a biological organism. Such a biological organism can utilize feedstock or biomass, such as sugars or carbohydrates obtained from an agricultural, plant, bacterial, or animal source.

As used herein, the term “biodegradable” refers to polymers of the present disclosure that decompose under natural conditions and are capable of being decomposed by living organisms.

As used herein, the term “centrifuge retention capacity” or “CRC” refers to the ability of a superabsorbent polymer, such as those described herein, to retain a liquid therein after being saturated and subjected to centrifugation under controlled conditions and is stated as grams of liquid retained per gram weight of the sample (g/g). The CRC value is determined herein using Iso 17190-6:2019 or a comparable assay. The test is performed under a 0.9% saline solution.

As used herein, the term “comprising” is intended to mean that the compositions and methods include the recited elements, but do not exclude others.

As used herein, the term “crosslinked” refers to a means for rendering water-soluble materials substantially water-insoluble but swellable, for example with the use of one or more crosslinking agents as disclosed herein. Without wishing to be bound by theory, crosslinking means can include, for example, physical entanglement, crystalline domains, covalent bonds, ionic complexes and associations, hydrophilic associations such as hydrogen bonding, hydrophobic associations, Van der Waals forces, or any combination thereof.

As used herein, the term “free swell capacity” or “FSC” refers to an amount in grams in which 1 gram of a dried polymer, such as the SAPs described herein can absorb a specified aqueous medium under a 0.9% saline solution. The test method for determination of the FSC in saline by gravimetric measurement is determined by ISO 17190-5:2020 or a comparable assay.

As used herein, the term “hydrogel” refers to an aqueous polymeric system of the SAPs described.

As used herein, the term “ISO” refers to International Standards Organization.

As used herein, the term “ISO 20200:2024” refers to a method of determining the degree of disintegration of plastic materials when exposed to a laboratory-scale composting environment as provided by the International Organization for Standardization.

As used herein, the term “superabsorbent” refers to polymers described herein that capable of absorbing a liquid, such as water, at a capacity of from at least 10 g/g and up to at least 1200 g/g.

As used herein, the term “white” is a measure which correlates the visual ratings of whiteness for the material, based on a “Whiteness Index” (WI). The terms “white” and “WI” refer to the measure of how closely a surface of a material, such as an SAP disclosed herein, matches the properties of a perfect reflecting diffuser (i.e., an ideal reflecting surface that neither absorbs nor transmits light but reflects it as equal intensities in all directions). The WI for calculating yellowness and whiteness indices from instrumentally measured color coordinates when used herein is based on measurement of uniform color spaces on the CIE 1976 L*a*b* scale for any illuminant as recommended by the Commission Internationale de l'Eclairage (CIE). The CIE L*a*b* scale is a simplified cube root version of the Adams-Nickerson space produced by the quantities of CIE L*a*b* in rectangular coordinates. The relationship of the CIE L*a*b* scale and the CIE XYZ scale for any illuminant is described in the ASTM E 308 Standard Practice for Computing the Colors of Objects by Using the CIE System. From the measurement of the quantities of CIE L*a*b* using a colorimeter, the WI in relationship with physical observation was derived as Whiteness Index=(L*/b*)−a*>7.5=Sample looks white, wherein where L* characterizes white and black, a* characterizes red and green, b* characterizes yellow (L*−=dark, L*+=white; b*−=blue, b*+=yellow, a*−=green, and a*+=red). Colorimeters are instruments that shine light on a sample and measure reflectance as a function of wave-length, and usually report color information in CIE color space. The theoretical “perfect white” has reference values of 100% across the visible spectrum with corresponding colorimetric values of L*=100.00, a*=0.00 and b*=0.00. An item near white, it may be darker, lower L* value, and possibly be slightly chromatic either in the red-green dimension (a*) of in the yellow-blue dimension (b*). Colorimetry testing in the present disclosure was conducted using Fru WR-10QC Portable Digital Colorimeter, operating in the CIELAB color space with a full spectrum LED D65 light source, 10° SCI, and 40 mm Integrating sphere.

### Overview

The present disclosure relates to superabsorbent polymers (SAPs), methods of making and using thereof. SAPs as described herein, are capable of absorbing and retaining, including under pressure, significant amounts of water or aqueous solutions.

The present disclosure describes polyaspartic acid (PASP) based SAPs and methods for the production of such PASP-based SAPs for use in the manufacture of biodegradable, absorbent articles, for example, substantially 100% biodegradable diapers. The present disclosure also describes polyaspartic acid (PASP) based SAPs and methods for the production of such PASP-based SAPs for use in the manufacture of biodegradable, absorbent articles, for example, 100% biodegradable diapers.

Polyaspartic acid has previously been investigated for use as an SAP. The biodegradability of PASP was also extensively investigated. A complex environment of factors are involved in degradation of the PASPs, including factors such as microorganisms, solar radiation, temperature, and moisture cycles. Key properties that influence biodegradability include chemical structure, molecular weight, morphology, crystallinity, glass transition temperatures, hydrophilicity, and water uptake abilities. For example, a sodium poly(aspartate) polymer synthesized with phosphoric acid as a catalyst is substantially 100% biodegradable, whereas a synthesis of the polymer without a catalyst yields a polymer with only 70% biodegradability. In some embodiments, a sodium poly(aspartate) polymer synthesized with phosphoric acid as a catalyst is 100% biodegradable, whereas a synthesis of the polymer without a catalyst yields a polymer with only 70% biodegradability. The biodegradation of PASP is influenced by branching structures and irregular end groups. Without wishing to be bound by theory, a linear poly(amide) backbone can be required for 100% biodegradation.

A crosslinked sodium salt form of poly(aspartic acid) (PASP), as described herein, was surprisingly found to be substantially 100% biodegradable. For example, in some embodiments, PASP is crosslinked with an amine crosslinker, using a new base and solvent system to produce a substantially 100% biodegradable SAP. In some embodiments, a crosslinked sodium salt form of poly(aspartic acid) (PASP), as described herein, was surprisingly found to be 100% biodegradable. In some embodiments, PASP is crosslinked with an amine crosslinker, using a new base and solvent system to produce a 100% biodegradable SAP.

The method of the present disclosure for forming biodegradable SAPs uses a mild base and a novel solvent system. In some embodiments, the biodegradable SAP is formed using a strong base, e.g., NaOH. In some embodiments, the base is a water-based imidazole buffer. In some embodiments, the amine crosslinker is 1,8-diaminooctane. In some embodiments, the amine crosslinker is cystamine. In some embodiments, the solvent is DMSO. In some embodiments, the solvent is diphenyl acetone (DPA). In some embodiments, the solvent is Cyrene. In some embodiments, the base is sodium carbonate (Na₂CO₃).

In some embodiments, the SAP of the present disclosure is biodegradable through home composting conditions of 25° C. for up to 180 days, with no more than 10% of original dry weight of material greater than 2 mm. In some embodiments, the SAP of the present disclosure has a water absorption in a range from 30 g/g to 350 g/g. In some embodiments, the SAP of the present disclosure has a water absorption of up to 1400 g/g. In some embodiments, the SAP of the present disclosure has a saline absorption in a range from 10 g/g to 115 g/g. In some embodiments, the SAP of the present disclosure has a free swell capacity in a range from 30 g/g to 40 g/g under 0.9% saline solution. In some embodiments, the SAP of the present disclosure has an absorption under load of 0.7 psi in a range from 5 g/g to 10 g/g. In some embodiments, the SAP of the present disclosure has an absorption under load of up to 20 g/g. In some embodiments, the SAP of the present disclosure has a centrifuge retention capacity in a range from 9 g/g to 35 g/g.

Described herein are biodegradable SAPs. In some embodiments, the biodegradable SAPs are prepared by using amino acid as a base material, with its amide bonds being more susceptible to degradation with microorganisms and enzymes, as compared to polysaccharide-based SAPs, which although bio-based are more difficult to biodegrade after crosslinking due to its hydroxyl groups being blocked during chemical modification, making it less accessible for enzymatic cleavage.

SAPs described herein have higher capacity for water uptake and swelling. Without wishing to be bound to theory, the present disclosure describes that the higher capacity for water uptake and swelling can be associated with longer polymer chains (higher Mw PSI), which can afford more extended polymer backbones and more entanglements (or network potential after hydrolysis/cross-linking), resulting in increased capacity for water uptake and swelling. Current industrial syntheses of PSI generally produce polymers with a weight-average molecular weight (Mw) in the region of 20,000-50,000 g/mol, depending on reaction parameters such as catalyst type/concentration, temperature, reaction time, and solvent system. For example, in an exemplary embodiment PSI are synthesized with molecular weights of about 64,300 g/mol using phosphoric acid and a mixed solvent (mesitylene/sulfolane) under optimized acid-catalyzed polycondensation conditions. In more routine or less optimized conditions (e.g., simple thermal polymerization with phosphate catalysis), PSI of lower Mw (≈20,000-25,000 g/mol) are also produced. In the present disclosure, it was surprisingly found that by optimizing key parameters (such as catalyst loading, temperature, solvent system etc.), it was possible to synthesize PSI with Mw >66,000 g/mol which is above the typical industrial range. Importantly, it was also found that this higher-Mw PSI precursor translates into PASP materials with markedly improved absorption performance, as described herein.

A surface crosslinking (or coating) was used to enhance mechanical strength and gel integrity of the disclosed polymers to keep them absorbent while under load and able to resist compression. Surface crosslinking agents are able to react with carboxyl groups near a surface of the polymer, forming additional crosslinks only in an outer shell, not throughout the whole polymer particle. Such surface cross linking was performed only on the surface of the polymer particles to limit excessive crosslinking within the polymer particles, to improve the absorption under load.

Uncoated polyaspartic acid SAPs, and uncoated crosslinked PASPs have a low AUL (9 g/g), which can be significantly improved by a surface crosslinking process as described herein, to result in coated SAPs with AUL values of more than double the value of the uncoated polymer, i.e., AUL of 16-30 g/g were achieved while keeping the CRC values at an industry acceptable level of 30 g/g. In some embodiments, the AUL is 18-25 g/g. In some embodiments, the AUL is 20-22 g/g. In some embodiments, the AUL is about 18 g/g, about 20 g/g, about 22 g/g, about 24 g/g, about 26 g/g, about 28 g/g, or about 30 g/g.

No existing publication was found on White color of PASP. While most commercially available PSIs are beige to brown in color and PASP synthesized using commercially available unpurified samples produce products that are beige to brown in color, it was surprisingly found that white SAPs may be formed by controlling the color of its PSI, which impacts the color of its PASP, by purifying the PSI before crosslinking. By generating a white to off white colored PSI after purification produces a white PASP sample. The present disclosure describes such an achievement through application of a vacuum during synthesis of its PSI, thereby avoiding the oxidation of the product, and then purifying it by reprecipitation.

### Crosslinkers

Described herein are SAPs that comprise amino acids crosslinked with one or more crosslinkers.

In some embodiments, the crosslinker comprises an amine based crosslinker. In some embodiments, the amine crosslinker comprises ethylenediamine; 1,4-butanediamine; 1,5-pentanediamine; 1,6-hexamethylenediamine; 1, 10-diaminodecane; 1,12-diaminododecane; L-lysine; L-ornithine; diethylenetriamine; triethylenetetramine; 1,4-diaminocyclohexane; p-phenylenediamine, or tris(2-aminoethyl)amine. In some embodiments, the crosslinker comprises an epoxide based crosslinker. In some embodiments, the epoxy crosslinker comprises ethylene glycol diglycidyl ether (EGDGE); poly(ethylene glycol) diglycidyl ether (PEGDGE); ethylene glycol; diethyleneglycol diglycidyl ether; 1,4-butanediol diglycidyl ether; glycerol polyglycidyl ether; sorbitol polyglycidyl ether; 2-oxazolidinone; N-(2-hydroxy ethyl)-2 oxazolidinone (HEONON); ethylene carbonate; 1,4 butanediol; or glycerol.

In some embodiments, the crosslinker disclosed herein is present at a concentration of from at least about 0.1 wt % to at least about 15 wt % of the SAP. In some embodiments, the crosslinker disclosed herein is present at a concentration of from at least about 1 wt % to at least about 10 wt % of the SAP. In some embodiments, the crosslinker disclosed herein is present at a concentration of from at least about 2 wt % to at least about 5 wt % of the SAP. In some embodiments, the crosslinker disclosed herein is present at a concentration of 2.5 wt % of the SAP. In some embodiments, the crosslinker disclosed herein is present at a concentration of at least about 0.1 wt %, at least about 0.5 wt %, at least about 1 wt %, at least about 1.5 wt %, at least about 2 wt %, at least about 2.5 wt %, at least about 3 wt %, at least about 3.5 wt %, at least about 4 wt %, at least about 4.5 wt %, at least about 5 wt %, at least about 5.5 wt %, at least about 6 wt %, at least about 6.5 wt %, at least about 7 wt %, at least about 7.5 wt %, at least about 8 wt %, at least about 8.5 wt %, at least about 9 wt %, at least about 9.5 wt %, at least about 10 wt %, at least about 10.5 wt %, at least about 11 wt %, at least about 11.5 wt %, at least about 12 wt %, at least about 12.5 wt %, at least about 13 wt %, at least about 13.5 wt %, at least about 14 wt %, at least about 14.5 wt %, or at least about 15 wt % of the SAP.

### Superabsorbent Polymers (SAPs) and Hydrogels

In some embodiments, the SAPs of the present disclosure comprise an amino acid polymer crosslinked with one or more crosslinkers described herein, and uses thereof.

In some embodiments, the superabsorbent polymer has a water absorption capacity of from 10 g/g to 600 g/g. In some embodiments, the superabsorbent polymer has a water absorption capacity of from 600 g/g to 1400 g/g. In some embodiments, the superabsorbent polymer has a water absorption capacity of from 20 g/g to 550 g/g. In some embodiments, the superabsorbent polymer has a water absorption capacity of from 30 g/g to 500 g/g. In some embodiments, the superabsorbent polymer has a water absorption capacity of about 40 g/g, about 50 g/g, about 60 g/g, about 70 g/g, about 80 g/g, about 90 g/g, about 100 g/g, about 110 g/g, about 120 g/g, about 130 g/g, about 140 g/g, about 150 g/g, about 160 g/g, about 170 g/g, about 180 g/g, about 190 g/g, about 200 g/g, about 210 g/g, about 220 g/g, about 230 g/g, about 240 g/g, about 250 g/g, about 260 g/g, about 270 g/g, about 280 g/g, about 290 g/g, about 300 g/g, about 310 g/g, about 320 g/g, about 330 g/g, about 340 g/g, about 350 g/g, about 360 g/g, about 370 g/g, about 380 g/g, about 390 g/g, about 400 g/g, about 410 g/g, about 420 g/g, about 430 g/g, about 440 g/g, about 450 g/g, about 460 g/g, about 470 g/g, about 480 g/g, about 490 g/g, or about 500 g/g. In some embodiments, the SAP has a water absorption capacity of from 700 g/g to 1400 g/g. In some embodiments, the SAP has a water absorption capacity of from 800 g/g to 1400 g/g. In some embodiments, the SAP has a water absorption capacity of from 900 g/g to 1400 g/g. In some embodiments, the SAP has a water absorption capacity of from 1000 g/g to 1400 g/g. In some embodiments, the SAP has a water absorption capacity of from 1100 g/g to 1400 g/g. In some embodiments, the SAP has a water absorption capacity of from 600 g/g to 1100 g/g. In some embodiments, the SAP has a water absorption capacity of from 600 g/g to 1000 g/g. In some embodiments, the SAP has a water absorption capacity of from 600 g/g to 900 g/g. In some embodiments, the SAP has a water absorption capacity of from 600 g/g to 800 g/g. In some embodiments, the SAP has a water absorption capacity of from 600 g/g to 700 g/g. In some embodiments, the SAP has a water absorption capacity of about 600 g/g, about 650 g/g, about 700 g/g, about 750 g/g, about 800 g/g, about 850 g/g, about 900 g/g, about 950 g/g, about 1000 g/g, about 1050 g/g, about 1100 g/g, about 1150 g/g, or about 1200 g/g.

In some embodiments, the SAP has a water absorption capacity of from at least about 10 g/g to at least about 600 g/g. In some embodiments, the SAP has a water absorption capacity of from at least about 20 g/g to at least about 550 g/g. In some embodiments, the SAP has a water absorption capacity of from at least about 30 g/g to at least about 500 g/g. In some embodiments, the SAP has a water absorption capacity at least about 40 g/g, at least about 50 g/g, at least about 60 g/g, at least about 70 g/g, at least about 80 g/g, at least about 90 g/g, at least about 100 g/g, at least about 110 g/g, at least about 120 g/g, at least about 130 g/g, at least about 140 g/g, at least about 150 g/g, at least about 160 g/g, at least about 170 g/g, at least about 180 g/g, at least about 190 g/g, at least about 200 g/g, at least about 210 g/g, at least about 220 g/g, at least about 230 g/g, at least about 240 g/g, at least about 250 g/g, at least about 260 g/g, at least about 270 g/g, at least about 280 g/g, at least about 290 g/g, at least about 300 g/g, at least about 310 g/g, at least about 320 g/g, at least about 330 g/g, at least about 340 g/g, at least about 350 g/g, at least about 360 g/g, at least about 370 g/g, at least about 380 g/g, at least about 390 g/g, at least about 400 g/g, at least about 410 g/g, at least about 420 g/g, at least about 430 g/g, at least about 440 g/g, at least about 450 g/g, at least about 460 g/g, at least about 470 g/g, at least about 480 g/g, at least about 490 g/g, or at least about 500 g/g.

In some embodiments, the SAP has a saline absorption of from 10 g/g to 100 g/g. In some embodiments, the SAPs have a saline absorption of from 20 g/g to 90 g/g. In some embodiments, the SAPs have a saline absorption of from 30 g/g to 80 g/g. In some embodiments, the SAPs have a saline absorption of from 40 g/g to 70 g/g. In some embodiments, the SAPs have a saline absorption of about 10 g/g, about 15 g/g, about 20 g/g, about 25 g/g, about 30 g/g, about 35 g/g, about 40 g/g, about 45 g/g, about 50 g/g, about 55 g/g, about 60 g/g, about 65 g/g, about 70 g/g, about 75 g/g, about 80 g/g, about 85 g/g, about 90 g/g, about 95 g/g, or about 100 g/g.

In some embodiments, the SAP has a saline absorption of from at least about 10 g/g to at least about 100 g/g. In some embodiments, the SAPs have a saline absorption of from at least about 20 g/g to at least about 90 g/g. In some embodiments, the SAPs have a saline absorption of from at least about 30 g/g to at least about 80 g/g. In some embodiments, the SAPs have a saline absorption of from at least about 40 g/g to at least about 70 g/g. In some embodiments, the SAPs have a saline absorption of at least about 10 g/g, at least about 15 g/g, at least about 20 g/g, at least about 25 g/g, at least about 30 g/g, at least about 35 g/g, at least about 40 g/g, at least about 45 g/g, at least about 50 g/g, at least about 55 g/g, at least about 60 g/g, at least about 65 g/g, at least about 70 g/g, at least about 75 g/g, at least about 80 g/g, at least about 85 g/g, at least about 90 g/g, at least about 95 g/g, or at least about 100 g/g.

In some embodiments, the SAP has an absorption under load (AUL) of from 1 g/g to 100 g/g under a load of 0.7 psi. In some embodiments, the SAPs have an AUL of from 10 g/g to 90 g/g under a load of 0.7 psi. In some embodiments, the SAPs have an AUL of from 20 g/g to 80 g/g under a load of 0.7 psi. In some embodiments, under a load of 0.7 psi, the SAPs have an AUL of about 1 g/g, about 5 g/g, about 10 g/g, about 15 g/g, about 20 g/g, about 25 g/g, about 30 g/g, about 35 g/g, about 40 g/g, about 45 g/g, about 50 g/g, about 55 g/g, about 60 g/g, about 65 g/g, about 70 g/g, about 75 g/g, about 80 g/g, about 85 g/g, about 90 g/g, about 95 g/g, or about 100 g/g.

In some embodiments, the SAP has an absorption under load (AUL) of from at least about 1 g/g to at least about 100 g/g under a load of 0.7 psi. In some embodiments, the SAPs have an AUL of from at least about 10 g/g to at least about 90 g/g under a load of 0.7 psi. In some embodiments, the SAPs have an AUL of from at least about 20 g/g to at least about 80 g/g under a load of 0.7 psi. In some embodiments, under a load of 0.7 psi, the SAPs have an AUL of at least about 1 g/g, at least about 5 g/g, at least about 10 g/g, at least about 15 g/g, at least about 20 g/g, at least about 25 g/g, at least about 30 g/g, at least about 35 g/g, at least about 40 g/g, at least about 45 g/g, at least about 50 g/g, at least about 55 g/g, at least about 60 g/g, at least about 65 g/g, at least about 70 g/g, at least about 75 g/g, at least about 80 g/g, at least about 85 g/g, at least about 90 g/g, at least about 95 g/g, or at least about 100 g/g.

In some embodiments, the SAP are subjected to analysis using an ISO 20200:2024 method to determine the degree of disintegration of plastic materials when exposed to a laboratory-scale composting environment. In some embodiments, the SAP is degraded upon hydrolysis at a temperature of 15° C. or higher in 20 days when measured by ISO 20200:2024 or a comparable assay. In some embodiments, the SAPs may be degraded up to about 75% or greater at a temperature of 15° C. in 20 days when measured by ISO 20200:2024 or a comparable assay. In some embodiments, the SAPs may be degraded up to about 80% or greater at a temperature of 15° C. in 20 days when measured by ISO 20200:2024 or a comparable assay. In some embodiments, the SAPs may be degraded up to about 85% or greater at a temperature of 15° C. in 20 days when measured by ISO 20200:2024 or a comparable assay. In some embodiments, the SAPs may be degraded up to about 90% or greater at a temperature of 15° C. in 20 days when measured by ISO 20200:2024 or a comparable assay. In some embodiments, the SAPs may be degraded up to about 95% or greater at a temperature of 15° C. in 20 days when measured by ISO 20200:2024 or a comparable assay. In some embodiments, the SAPs may be degraded up to about 99% or greater at a temperature of 25° C. in 20 days when measured by ISO 20200:2024 or a comparable assay. In some embodiments, the SAPs may be degraded up to about 100% at a temperature of 15° C. in 20 days when measured by ISO 20200:2024 or a comparable assay. In some embodiments, the SAP is degraded in 20 days when measured by ISO 20200:2024 or a comparable assay at temperature of about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., or about 45° C.

In some embodiments, the SAP are subjected to analysis using an ISO 20200:2024 method to determine the degree of disintegration of plastic materials when exposed to a laboratory-scale composting environment. In some embodiments, the SAP is degraded upon hydrolysis at a temperature of 15° C. or higher in 20 days when measured by ISO 20200:2024 or a comparable assay. In some embodiments, the SAPs may be degraded up to at least about 75% or greater at a temperature of 15° C. in 20 days when measured by ISO 20200:2024 or a comparable assay. In some embodiments, the SAPs may be degraded up to at least about 80% or greater at a temperature of 15° C. in 20 days when measured by ISO 20200:2024 or a comparable assay. In some embodiments, the SAPs may be degraded up to at least about 85% or greater at a temperature of 15° C. in 20 days when measured by ISO 20200:2024 or a comparable assay. In some embodiments, the SAPs may be degraded up to at least about 90% or greater at a temperature of 15° C. in 20 days when measured by ISO 20200:2024 or a comparable assay. In some embodiments, the SAPs may be degraded up to at least about 95% or greater at a temperature of 15° C. in 20 days when measured by ISO 20200:2024 or a comparable assay. In some embodiments, the SAPs may be degraded up to at least about 99% or greater at a temperature of 25° C. in 20 days when measured by ISO 20200:2024 or a comparable assay. In some embodiments, the SAPs may be degraded up to at least about 100% at a temperature of 15° C. in 20 days when measured by ISO 20200:2024 or a comparable assay. In some embodiments, the SAP is degraded in 20 days when measured by ISO 20200:2024 or a comparable assay at temperature of at least about 15° C., at least about 16° C., at least about 17° C., at least about 18° C., at least about 19° C., at least about 20° C., at least about 21° C., at least about 22° C., at least about 23° C., at least about 24° C., at least about 25° C., at least about 26° C., at least about 27° C., at least about 28° C., at least about 29° C., at least about 30° C., at least about 31° C., at least about 32° C., at least about 33° C., at least about 34° C., at least about 35° C., at least about 36° C., at least about 37° C., at least about 38° C., at least about 39° C., at least about 40° C., at least about 41° C., at least about 42° C., at least about 43° C., at least about 44° C., or at least about 45° C.

In some embodiments, the SAP is degraded in about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, or about 25 days.

In some embodiments, the SAP is degraded in at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, or at least about 25 days.

In some embodiments, the SAPs are degraded in an amount of about 60%, about 62%, about 64%, about 66%, about 68%, about 70%, about 72%, about 74%, about 76%, about 78%, about 80%, about 82%, about 84%, about 86%, about 88%, about 90%, about 92%, about 94%, about 96%, about 98%, or about 100% of the SAP.

In some embodiments, the SAPs are degraded in an amount of at least about 60%, at least about 62%, at least about 64%, at least about 66%, at least about 68%, at least about 70%, at least about 72%, at least about 74%, at least about 76%, at least about 78%, at least about 80%, at least about 82%, at least about 84%, at least about 86%, at least about 88%, at least about 90%, at least about 92%, at least about 94%, at least about 96%, at least about 98%, or at least about 100% of the SAP.

Free swell capacity (FSC) described herein may be determined using an ISO 17190-5: 2020 (E) protocol. In some embodiments, the SAPs comprise a free swell capacity (FSC) of from 20 g/g to 100 g/g under a 0.9% saline solution. In some embodiments, the SAPs comprise an FSC of from 30 g/g to 90 g/g under a 0.9% saline solution. In some embodiments, the SAPs comprise an FSC of from 40 g/g to 80 g/g under a 0.9% saline solution. In some embodiments, the SAP, under a 0.9% saline solution, have an FSC of about 20 g/g, about 25 g/g, about 30 g/g, about 35 g/g, about 40 g/g, about 45 g/g, about 50 g/g, about 55 g/g, about 60 g/g, about 65 g/g, about 70 g/g, about 75 g/g, about 80 g/g, about 85 g/g, about 90 g/g, about 95 g/g, or about 100 g/g.

In some embodiments, the SAPs comprise a free swell capacity (FSC) of from at least about 20 g/g to at least about 100 g/g under a 0.9% saline solution. In some embodiments, the SAPs comprise an FSC of from at least about 30 g/g to at least about 90 g/g under a 0.9% saline solution. In some embodiments, the SAPs comprise an FSC of from at least about 40 g/g to at least about 80 g/g under a 0.9% saline solution. In some embodiments, the SAP, under a 0.9% saline solution, have an FSC of at least about 20 g/g, at least about 25 g/g, at least about 30 g/g, at least about 35 g/g, at least about 40 g/g, at least about 45 g/g, at least about 50 g/g, at least about 55 g/g, at least about 60 g/g, at least about 65 g/g, at least about 70 g/g, at least about 75 g/g, at least about 80 g/g, at least about 85 g/g, at least about 90 g/g, at least about 95 g/g, or at least about 100 g/g.

Centrifuge retention capacity (CRC) values described herein are determined using an ISO/DIS 17190-6: 2019 (E) and EDENA-NWSP 241.0.R2 (15) protocols. In some embodiments, the SAPs comprise a centrifuge retention capacity (CRC) of from 10 g/g to 50 g/g. In some embodiments, the SAPs comprise a CRC of from 20 g/g to 45 g/g. In some embodiments, the SAPs comprise a CRC of from 25 g/g to 40 g/g. In some embodiments, the SAPs have a CRC of about 10 g/g, about 15 g/g, about 20 g/g, about 25 g/g, about 30 g/g, about 35 g/g, about 40 g/g, about 45 g/g, or about 50 g/g.

In some embodiments, the SAPs comprise a centrifuge retention capacity (CRC) of from at least about 10 g/g to at least about 50 g/g. In some embodiments, the SAPs comprise a CRC of from at least about 20 g/g to at least about 45 g/g. In some embodiments, the SAPs comprise a CRC of from at least about 25 g/g to at least about 40 g/g. In some embodiments, the SAPs have a CRC of at least about 10 g/g, at least about 15 g/g, at least about 20 g/g, at least about 25 g/g, at least about 30 g/g, at least about 35 g/g, at least about 40 g/g, at least about 45 g/g, or at least about 50 g/g.

In some embodiments, the SAP comprises biobased carbons, such as carbons derived from or synthesized by a biological organism, e.g., a renewable resource like a feedstock or biomass. In some embodiments, the SAPs comprise 50% or more biobased carbons. In some embodiments, the SAPs comprise 65% or more biobased carbons. In some embodiments, the SAPs comprise 80% or more biobased carbons. In some embodiments, the SAPs contain biobased carbons in an amount of about 50%, about 55%, about 60%, about 65%, about 70%, about 75%, about 80%, about 85%, about 90%, about 95%, or about 100%.

In some embodiments, the SAPs of the present disclosure comprise a poly(aspartic acid) polymer and one or more crosslinking agents. In some embodiments, the one or more crosslinking agents is present at a concentration of from 0.1 wt % to 15 wt % of the SAP. In some embodiments, the one or more crosslinking agents is present at a concentration of from 0.1 wt % to 15 wt % of the SAP. In some embodiments, the one or more crosslinking agents is present at a concentration of from 1 wt % to 10 wt % of the SAP. In some embodiments, the one or more crosslinking agents is present at a concentration of from 2 wt % to 5 wt % of the SAP. In some embodiments, the one or more crosslinking agents is present at a concentration of 2.5 wt % of the SAP. In some embodiments, the one or more crosslinking agents is present at a concentration of about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 10.5 wt %, about 11 wt %, about 11.5 wt %, about 12 wt %, about 12.5 wt %, about 13 wt %, about 13.5 wt %, about 14 wt %, about 14.5 wt %, or about 15 wt % of the SAP.

In some embodiments, each of the one or more crosslinking agents is independently present at a concentration of from 0.1 wt % to 15 wt % of the SAP. In some embodiments, each of the one or more crosslinking agents is independently present at a concentration of from 0.1 wt % to 15 wt % of the SAP. In some embodiments, each of the one or more crosslinking agents is independently present at a concentration of from 1 wt % to 10 wt % of the SAP. In some embodiments, each of the one or more crosslinking agents is independently present at a concentration of from 2 wt % to 5 wt % of the SAP. In some embodiments, each of the one or more crosslinking agents is independently present at a concentration of 2.5 wt % of the SAP. In some embodiments, each of the one or more crosslinking agents is independently present at a concentration of about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 10.5 wt %, about 11 wt %, about 11.5 wt %, about 12 wt %, about 12.5 wt %, about 13 wt %, about 13.5 wt %, about 14 wt %, about 14.5 wt %, or about 15 wt % of the SAP.

In some embodiments, the SAPs of the present disclosure comprise a poly(aspartic acid) polymer crosslinked with 1,8-diaminooctane, or use thereof. In some embodiments, SAPs provided herein comprises a poly(aspartic acid) polymer crosslinked with 1,8-diaminooctane and cystamine. In some embodiments, the SAPs of the present disclosure comprise a poly(aspartic acid) polymer crosslinked with 1,6-hexamethylenediamine. In some embodiments, the 1,8-diaminooctane is present at a concentration of from 0.1 wt % to 15 wt % of the SAP. In some embodiments, the 1,8-diaminooctane is present at a concentration of from 1 wt % to 10 wt % of the SAP. In some embodiments, the 1,8-diaminooctane is present at a concentration of from 2 wt % to 5 wt % of the SAP. In some embodiments, the 1,8-diaminooctane is present at a concentration of 2.5 wt % of the SAP. In some embodiments, the 1,6-hexamethylenediamine is present at a concentration of from 0.1 wt % to 15 wt % of the SAP. In some embodiments, the 1,6-hexamethylenediamine is present at a concentration of from 1 wt % to 10 wt % of the SAP. In some embodiments, the 1,6-hexamethylenediamine is present at a concentration of from 2 wt % to 5 wt % of the SAP. In some embodiments, the 1,6-hexamethylenediamine is present at a concentration of 2.5 wt % of the SAP. In some embodiments, the 1,8-diaminooctane is present at a concentration of about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 10.5 wt %, about 11 wt %, about 11.5 wt %, about 12 wt %, about 12.5 wt %, about 13 wt %, about 13.5 wt %, about 14 wt %, about 14.5 wt %, or about 15 wt % of the SAP. In some embodiments, the 1,6-hexamethylenediamine is present at a concentration of about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, about 10 wt %, about 10.5 wt %, about 11 wt %, about 11.5 wt %, about 12 wt %, about 12.5 wt %, about 13 wt %, about 13.5 wt %, about 14 wt %, about 14.5 wt %, or about 15 wt % of the SAP.

In some embodiments, the 1,8-diaminooctane is present at a concentration of from at least about 0.1 wt % to at least about 15 wt % of the SAP. In some embodiments, the 1,8-diaminooctane is present at a concentration of from at least about 1 wt % to at least about 10 wt % of the SAP. In some embodiments, the 1,8-diaminooctane is present at a concentration of from at least about 2 wt % to at least about 5 wt % of the SAP. In some embodiments, the 1,8-diaminooctane is present at a concentration of 2.5 wt % of the SAP. In some embodiments, the 1,8-diaminooctane is present at a concentration of at least about 0.1 wt %, at least about 0.5 wt %, at least about 1 wt %, at least about 1.5 wt %, at least about 2 wt %, at least about 2.5 wt %, at least about 3 wt %, at least about 3.5 wt %, at least about 4 wt %, at least about 4.5 wt %, at least about 5 wt %, at least about 5.5 wt %, at least about 6 wt %, at least about 6.5 wt %, at least about 7 wt %, at least about 7.5 wt %, at least about 8 wt %, at least about 8.5 wt %, at least about 9 wt %, at least about 9.5 wt %, at least about 10 wt %, at least about 10.5 wt %, at least about 11 wt %, at least about 11.5 wt %, at least about 12 wt %, at least about 12.5 wt %, at least about 13 wt %, at least about 13.5 wt %, at least about 14 wt %, at least about 14.5 wt %, or at least about 15 wt % of the SAP.

In some embodiments, the cystamine is present at a concentration of from 0.1 wt % to 10 wt % of the SAP. In some embodiments, the cystamine is present at a concentration of from 1 wt % to 5 wt % of the SAP. In some embodiments, the cystamine is present at a concentration of from 2 wt % to 4 wt % of the SAP. In some embodiments, the cystamine is present at a concentration of 2.5 wt % of the SAP. In some embodiments, the cystamine is present at a concentration of about 0.1 wt %, about 0.5 wt %, about 1 wt %, about 1.5 wt %, about 2 wt %, about 2.5 wt %, about 3 wt %, about 3.5 wt %, about 4 wt %, about 4.5 wt %, about 5 wt %, about 5.5 wt %, about 6 wt %, about 6.5 wt %, about 7 wt %, about 7.5 wt %, about 8 wt %, about 8.5 wt %, about 9 wt %, about 9.5 wt %, or about 10 wt % of the SAP.

In some embodiments, the cystamine is present at a concentration of from at least about 0.1 wt % to at least about 10 wt % of the SAP. In some embodiments, the cystamine is present at a concentration of from at least about 1 wt % to at least about 5 wt % of the SAP. In some embodiments, the cystamine is present at a concentration of from at least about 2 wt % to at least about 4 wt % of the SAP. In some embodiments, the cystamine is present at a concentration of 2.5 wt % of the SAP. In some embodiments, the cystamine is present at a concentration of at least about 0.1 wt %, at least about 0.5 wt %, at least about 1 wt %, at least about 1.5 wt %, at least about 2 wt %, at least about 2.5 wt %, at least about 3 wt %, at least about 3.5 wt %, at least about 4 wt %, at least about 4.5 wt %, at least about 5 wt %, at least about 5.5 wt %, at least about 6 wt %, at least about 6.5 wt %, at least about 7 wt %, at least about 7.5 wt %, at least about 8 wt %, at least about 8.5 wt %, at least about 9 wt %, at least about 9.5 wt %, or at least about 10 wt % of the SAP.

In some embodiments, the biodegradable poly(aspartic acid) hydrogels comprise a superabsorbent biopolymer comprising a poly(aspartic acid) polymer crosslinked with 1,8-diaminooctane and/or cystamine; and one or more fluids, a charged species, a biological molecule, an organic compound, a metal, or combinations thereof; wherein the SAP has a water absorption capacity of from 10 g/g to 600 g/g, and wherein the biodegradable poly(aspartic acid) hydrogel is degraded upon hydrolysis at a temperature of 15° C. in 20 days when measured by ISO 20200:2024 or a comparable assay.

In some embodiments, the biodegradable poly(aspartic acid) hydrogels comprise a superabsorbent biopolymer comprising a poly(aspartic acid) polymer crosslinked with 1,8-diaminooctane and/or cystamine; and one or more fluids, a charged species, a biological molecule, an organic compound, a metal, or combinations thereof; wherein the SAP has a water absorption capacity of from at least about 10 g/g to at least about 600 g/g, and wherein the biodegradable poly(aspartic acid) hydrogel is degraded upon hydrolysis at a temperature of 15° C. in 20 days when measured by ISO 20200:2024 or a comparable assay.

In some embodiments, in the biodegradable poly(aspartic acid) hydrogels, the SAP binds, adsorbs, absorbs, dissolves, sequesters, chelates, or any combination thereof the one or more fluids, the charged species, the biological molecule, the organic compound, the metal, or any combination thereof. In some embodiments, the SAP binds the one or more fluids, the charged species, the biological molecule, the organic compound, the metal, or any combination thereof. In some embodiments, the SAP adsorbs the one or more fluids, the charged species, the biological molecule, the organic compound, the metal, or any combination thereof. In some embodiments, the SAP absorbs the one or more fluids, the charged species, the biological molecule, the organic compound, the metal, or any combination thereof. In some embodiments, the SAP dissolves the one or more fluids, the charged species, the biological molecule, the organic compound, the metal, or any combination thereof. In some embodiments, the SAP sequesters the one or more fluids, the charged species, the biological molecule, the organic compound, the metal, or any combination thereof. In some embodiments, the SAP chelates the one or more fluids, the charged species, the biological molecule, the organic compound, the metal, or any combination thereof.

In some embodiments, the biodegradable poly(aspartic acid) hydrogels comprise one or more fluids comprising water, hydrocarbon, oil, alcohol, an aqueous solution, a non-aqueous solution, an ionic solution, a biological fluid, a gas, a waste-water, a fracking fluid, or any combination thereof. In some embodiments, the one or more fluids comprises water. In some embodiments, the one or more fluids comprises hydrocarbon. In some embodiments, the one or more fluids comprises oil. In some embodiments, the one or more fluids comprises alcohol. In some embodiments, the one or more fluids comprises an aqueous solution. In some embodiments, the one or more fluids comprises a non-aqueous solution. In some embodiments, the one or more fluids comprises an ionic solution. In some embodiments, the one or more fluids comprises a biological fluid. In some embodiments, the one or more fluids comprises a gas. In some embodiments, the one or more fluids comprises a waste-water. In some embodiments, the one or more fluids comprises a fracking fluid.

In some embodiments, the biodegradable poly(aspartic acid) hydrogels comprise biological fluid comprising blood, urine, fecal water, vomit, or any combination thereof. In some embodiments, the biological fluid comprises blood. In some embodiments, the biological fluid comprises urine. In some embodiments, the biological fluid comprises fecal water. In some embodiments, the biological fluid comprises vomit.

In some embodiments, the biodegradable poly(aspartic acid) hydrogels comprise a biological molecule comprising a peptide, a polypeptide, a nucleic acid, an oligonucleotide, a carbohydrate, a lipid, or any combination thereof. In some embodiments, the biological molecule comprises a peptide. In some embodiments, the biological molecule comprises a polypeptide. In some embodiments, the biological molecule comprises a nucleic acid. In some embodiments, the biological molecule comprises an oligonucleotide. In some embodiments, the biological molecule comprises a carbohydrate. In some embodiments, the biological molecule comprises a lipid.

In some embodiments, the biodegradable poly(aspartic acid) hydrogels comprise potassium ions (K⁺), calcium ions (Ca²⁺), sodium ions (Na⁺), chloride ions (Cl⁻), fluoride ions (F⁻), phosphite ions (PO₃³⁻), sulfate ions (SO₄²⁻), sulfite ions (SO₃²⁻), phosphate ions (PO₄³⁻), polyatomic ions, metal ions, charged biological molecules, or any combination thereof. In some embodiments, the charged species comprises potassium ions (K⁺). In some embodiments, the charged species comprises calcium ions (Ca²⁺). In some embodiments, the charged species comprises sodium ions (Na⁺). In some embodiments, the charged species comprises chloride ions (Cl⁻). In some embodiments, the charged species comprises fluoride ions (F⁻). In some embodiments, the charged species comprises phosphite ions (PO₃³⁻). In some embodiments, the charged species comprises sulfate ions (SO₄²⁻). In some embodiments, the charged species comprises sulfite ions (SO₃²⁻). In some embodiments, the charged species comprises phosphate ions (PO₄³⁻). In some embodiments, the charged species comprises polyatomic ions. In some embodiments, the charged species comprises metal ions. In some embodiments, the charged species comprises charged biological molecules.

In some embodiments, the biodegradable poly(aspartic acid) hydrogels comprise a metal comprising lead, mercury, cadmium, arsenic, copper, chromium, thallium, selenium, zinc, calcium, magnesium, silver, boron, lithium, ammonium, barium, strontium, manganese, silver, cesium, zinc, cadmium, selenium, calcium, magnesium, iron, radium, mercury, copper, lead, nickel, chromium, arsenic, gold, uranium, a carbonate of any of the foregoing, a sulphate of any of the foregoing, and/or phosphate of any of the foregoing, or any combination thereof. In some embodiments, the metal comprises lead. In some embodiments, the metal comprises mercury. In some embodiments, the metal comprises cadmium. In some embodiments, the metal comprises arsenic. In some embodiments, the metal comprises copper. In some embodiments, the metal comprises chromium. In some embodiments, the metal comprises thallium. In some embodiments, the metal comprises selenium. In some embodiments, the metal comprises zinc. In some embodiments, the metal comprises calcium. In some embodiments, the metal comprises magnesium. In some embodiments, the metal comprises silver. In some embodiments, the metal comprises boron. In some embodiments, the metal comprises lithium. In some embodiments, the metal comprises ammonium. In some embodiments, the metal comprises barium. In some embodiments, the metal comprises strontium. In some embodiments, the metal comprises manganese. In some embodiments, the metal comprises silver. In some embodiments, the metal comprises cesium. In some embodiments, the metal comprises zinc. In some embodiments, the metal comprises cadmium. In some embodiments, the metal comprises selenium. In some embodiments, the metal comprises calcium. In some embodiments, the metal comprises magnesium. In some embodiments, the metal comprises iron. In some embodiments, the metal comprises radium. In some embodiments, the metal comprises mercury. In some embodiments, the metal comprises copper. In some embodiments, the metal comprises lead. In some embodiments, the metal comprises nickel. In some embodiments, the metal comprises chromium. In some embodiments, the metal comprises arsenic. In some embodiments, the metal comprises gold. In some embodiments, the metal comprises uranium. In some embodiments, the metal comprises a carbonate of any of the foregoing. In some embodiments, the metal comprises a sulphate of any of the foregoing. In some embodiments, the metal comprises a phosphate of any of the foregoing.

### Polymer Degradation and Polymer Hydrolysis

In some embodiments, the SAP is degradable upon exposure to solar radiation. In some embodiments, the solar radiation comprises gamma rays, UV rays, visible light, infrared rays, x-rays, microwave radiation, radio waves, or any combination thereof. In some embodiments, the solar radiation comprises gamma rays. In some embodiments, the solar radiation comprises UV rays. In some embodiments, the solar radiation comprises visible light. In some embodiments, the solar radiation comprises infrared rays. In some embodiments, the solar radiation comprises x-rays. In some embodiments, the solar radiation comprises microwave radiation. In some embodiments, the solar radiation comprises radio waves.

In some embodiments, the SAP degrades upon contact with an effective amount of environmental moisture. In some embodiments, the environmental moisture comprises a body of water, soil moisture, waste moisture, or an atmospheric humidity. In some embodiments, the environmental moisture comprises a body of water. In some embodiments, the environmental moisture comprises soil moisture. In some embodiments, the environmental moisture comprises waste moisture. In some embodiments, the environmental moisture comprises an atmospheric humidity.

In some embodiments, the environmental moisture comprises a relative humidity of 10% to 60% or a moisture content of from 10% to 65%, or greater. In some embodiments, the environmental moisture comprises a relative humidity of about 10%, about 15%, about 20%, about 25%, about 30%, about 35%, about 40%, about 45%, about 50%, about 55%, or about 60%. In some embodiments, the environmental moisture comprises a moisture content of about 10%, about 15%, about 20%, about 25%, about 30%, about 35%, about 40%, about 45%, about 50%, about 55%, about 60%, or about 65%.

In some embodiments, the environmental moisture comprises a relative humidity of from at least about 10% to at least about 60% or a moisture content of from at least about 10% to at least about 65%, or greater. In some embodiments, the environmental moisture comprises a relative humidity of at least about 10%, at least about 15%, at least about 20%, at least about 25%, at least about 30%, at least about 35%, at least about 40%, at least about 45%, at least about 50%, at least about 55%, or at least about 60%. In some embodiments, the environmental moisture comprises a moisture content of at least about 10%, at least about 15%, at least about 20%, at least about 25%, at least about 30%, at least about 35%, at least about 40%, at least about 45%, at least about 50%, at least about 55%, at least about 60%, or at least about 65%.

In some embodiments, the SAP is hydrolyzed on exposure to an enzymatic hydrolysis or chemical hydrolysis. In some embodiments, the enzymatic hydrolysis occurs upon contact of the SAP with one or more enzymes or one or more microorganisms. In some embodiments, the one or more enzymes comprises one or more lipolytic enzymes, one or more proteolytic enzymes, one or more amylolytic enzymes, one or more cellulolytic enzymes, or any combination thereof. In some embodiments, the one or more enzymes comprises one or more lipolytic enzymes. In some embodiments, the one or more enzymes comprises one or more proteolytic enzymes. In some embodiments, the one or more enzymes comprises one or more amylolytic enzymes. In some embodiments, the one or more enzymes comprises one or more cellulolytic enzymes.

In some embodiments, the one or more lipolytic enzymes comprises one or more carboxylesterases and/or lipases. In some embodiments, the one or more lipolytic enzymes comprises one or more carboxylesterases. In some embodiments, the one or more lipolytic enzymes comprises one or more lipases.

In some embodiments, the one or more proteolytic enzymes comprises one or more serine proteases, cysteine proteases, threonine proteases, aspartic proteases, metalloproteases, glutamic proteases, or any combination thereof. In some embodiments, the one or more proteolytic enzymes comprises one or more serine proteases. In some embodiments, the one or more proteolytic enzymes comprises cysteine proteases. In some embodiments, the one or more proteolytic enzymes comprises threonine proteases. In some embodiments, the one or more proteolytic enzymes comprises aspartic proteases. In some embodiments, the one or more proteolytic enzymes comprises metalloproteases. In some embodiments, the one or more proteolytic enzymes comprises glutamic proteases.

In some embodiments, the one or more serine proteases comprises one or more trypsins, chymotrypsins, thrombins, subtilisins, or any combination thereof. In some embodiments, the one or more serine proteases comprises one or more trypsins. In some embodiments, the one or more serine proteases comprises one or more chymotrypsins. In some embodiments, the one or more serine proteases comprises one or more thrombins. In some embodiments, the one or more serine proteases comprises one or more subtilisins.

In some embodiments, the one or more amylolytic enzymes comprise one or more α-amylases, β-amylases, glucoamylases, or any combination thereof. In some embodiments, the one or more amylolytic enzymes comprise one or more α-amylases. In some embodiments, the one or more amylolytic enzymes comprise one or more β-amylases. In some embodiments, the one or more amylolytic enzymes comprise one or more glucoamylases.

In some embodiments, the one or more microorganisms comprise a bacterium or a yeast. In some embodiments, the one or more microorganisms comprises a bacterium. In some embodiments, the one or more microorganisms comprise a yeast.

In some embodiments, the one or more microorganisms comprise psychrophiles, mesophiles, thermophiles, actinomycetes, saprophytes, Absidia, Acremonium, Alternaria, amerospore, Arthrinium, ascospore, Aspergillus, Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus nidulans, Aspergillus ochraceus, Aspergillus oryzae, Aspergillus parasiticus, Aspergillus penicilloides, Aspergillus restrictus, Aspergillus sydowii, Aspergillus terreus, Aspergillus ustus, Aspergillus versicolor, Aspergillus/Penicillium-like, Aureobasidium, basidiomycetes, basidiospore, Bipolaris, Blastomyces, B. borstelensis, Botrytis, Candida, Cephalosporium, Chaetomium, Cladosporium, Cladosporium fulvum, Cladosporium herbarum, Cladosporium macrocarpum, Cladosporium sphaerospermum, conidia, conidium, conidobolus, Cryptococcus neoformans, Cryptostroma corticale, Cunninghamella, Curvularia, dreschlera, Epicoccum, Epidermophyton, Fungus, Fusarium, Fusarium solani, Geotrichum, Gliocladium, Helicomyces, Helminthosporium, Histoplasma, humicula, Hyaline mycelia, Memnoniella, Microsporum, mold, Monilia, Mucor, mycelium, myxomycetes, Nigrospora, Oidium, Paecilomyces, papulospora, Penicillium, Periconia, perithecium, Peronospora, phaeohyphomycosis, Phoma, Pithomyces, RhizoMucor, Rhizopus, Rhodococcus, Rhodotorula, rusts, Saccharomyces, Scopulariopsis, Sepedonium, Serpula lacrymans, smuts, Spegazzinia, spore, Sporoschisma, Sporothrix, Sporotrichum, Stachybotrys, Stemphylium, Syncephalastrum, Thermononespore fusca DSM43793, Torula, Trichocladium, Trichoderma, Trichophyton, Trichothecium, tritirachium, Ulocladium, Verticillium, Wallemia, yeast, or any combination thereof.

In some embodiments, the one or more microorganisms comprise psychrophiles. In some embodiments, the one or more microorganisms comprises mesophiles. In some embodiments, the one or more microorganisms comprise thermophiles. In some embodiments, the one or more microorganisms comprise actinomycetes. In some embodiments, the one or more microorganisms comprises saprophytes. In some embodiments, the one or more microorganisms comprise Absidia. In some embodiments, the one or more microorganisms comprise Acremonium. In some embodiments, the one or more microorganisms comprise Alternaria. In some embodiments, the one or more microorganisms comprise amerospore. In some embodiments, the one or more microorganisms comprise Arthrinium. In some embodiments, the one or more microorganisms comprise ascospore, Aspergillus. In some embodiments, the one or more microorganisms comprise Aspergillus caesiellus. In some embodiments, the one or more microorganisms comprise Aspergillus candidus. In some embodiments, the one or more microorganisms comprise Aspergillus carneus. In some embodiments, the one or more microorganisms comprise Aspergillus clavatus. In some embodiments, the one or more microorganisms comprise Aspergillus deflectus. In some embodiments, the one or more microorganisms comprise Aspergillus flavus. In some embodiments, the one or more microorganisms comprise Aspergillus fumigatus. In some embodiments, the one or more microorganisms comprise Aspergillus glaucus. In some embodiments, the one or more microorganisms comprise Aspergillus nidulans. In some embodiments, the one or more microorganisms comprise Aspergillus ochraceus. In some embodiments, the one or more microorganisms comprise Aspergillus oryzae. In some embodiments, the one or more microorganisms comprise Aspergillus parasiticus. In some embodiments, the one or more microorganisms comprise Aspergillus penicilloides. In some embodiments, the one or more microorganisms comprise Aspergillus restrictus. In some embodiments, the one or more microorganisms comprise Aspergillus sydowii. In some embodiments, the one or more microorganisms comprise Aspergillus terreus. In some embodiments, the one or more microorganisms comprise Aspergillus ustus. In some embodiments, the one or more microorganisms comprise Aspergillus versicolor. In some embodiments, the one or more microorganisms comprise Aspergillus/Penicillium-like, Aureobasidium. In some embodiments, the one or more microorganisms comprise basidiomycetes. In some embodiments, the one or more microorganisms comprise basidiospore. In some embodiments, the one or more microorganisms comprise Bipolaris. In some embodiments, the one or more microorganisms comprise Blastomyces. In some embodiments, the one or more microorganisms comprise B. borstelensis. In some embodiments, the one or more microorganisms comprise Botrytis. In some embodiments, the one or more microorganisms comprise Candida. In some embodiments, the one or more microorganisms comprise Cephalosporium. In some embodiments, the one or more microorganisms comprise Chaetomium. In some embodiments, the one or more microorganisms comprise Cladosporium. In some embodiments, the one or more microorganisms comprise Cladosporium fulvum. In some embodiments, the one or more microorganisms comprise Cladosporium herbarum. In some embodiments, the one or more microorganisms comprise Cladosporium macrocarpum. In some embodiments, the one or more microorganisms comprise Cladosporium sphaerospermum. In some embodiments, the one or more microorganisms comprise conidia, conidium. In some embodiments, the one or more microorganisms comprise conidobolus. In some embodiments, the one or more microorganisms comprise Cryptococcus neoformans. In some embodiments, the one or more microorganisms comprise cryptostroma corticale. In some embodiments, the one or more microorganisms comprise Cunninghamella. In some embodiments, the one or more microorganisms comprise Curvularia. In some embodiments, the one or more microorganisms comprise dreschlera. In some embodiments, the one or more microorganisms comprise Epicoccum, Epidermophyton. In some embodiments, the one or more microorganisms comprise Fungus, Fusarium. In some embodiments, the one or more microorganisms comprises Fusarium solani. In some embodiments, the one or more microorganisms comprise Geotrichum, Gliocladium. In some embodiments, the one or more microorganisms comprise Helicomyces. In some embodiments, the one or more microorganisms comprise Helminthosporium. In some embodiments, the one or more microorganisms comprise Histoplasma. In some embodiments, the one or more microorganisms comprise humicula. In some embodiments, the one or more microorganisms comprise Hyaline mycelia. In some embodiments, the one or more microorganisms comprise Memnoniella. In some embodiments, the one or more microorganisms comprise Microsporum. In some embodiments, the one or more microorganisms comprise mold, Monilia. In some embodiments, the one or more microorganisms comprise Mucor. In some embodiments, the one or more microorganisms comprise mycelium. In some embodiments, the one or more microorganisms comprise myxomycetes. In some embodiments, the one or more microorganisms comprise Nigrospora. In some embodiments, the one or more microorganisms comprise Oidium. In some embodiments, the one or more microorganisms comprise Paecilomyces. In some embodiments, the one or more microorganisms comprise papulospora. In some embodiments, the one or more microorganisms comprise Penicillium, Periconia. In some embodiments, the one or more microorganisms comprise perithecium. In some embodiments, the one or more microorganisms comprise Peronospora. In some embodiments, the one or more microorganisms comprise phaeohyphomycosis. In some embodiments, the one or more microorganisms comprise Phoma. In some embodiments, the one or more microorganisms comprises Pithomyces. In some embodiments, the one or more microorganisms comprise RhizoMucor. In some embodiments, the one or more microorganisms comprise Rhizopus. In some embodiments, the one or more microorganisms comprise Rhodococcus. In some embodiments, the one or more microorganisms comprise Rhodotorula. In some embodiments, the one or more microorganisms comprise rusts. In some embodiments, the one or more microorganisms comprise Saccharomyces. In some embodiments, the one or more microorganisms comprise Scopulariopsis. In some embodiments, the one or more microorganisms comprise Sepedonium. In some embodiments, the one or more microorganisms comprise Serpula lacrymans. In some embodiments, the one or more microorganisms comprise smuts. In some embodiments, the one or more microorganisms comprise Spegazzinia. In some embodiments, the one or more microorganisms comprise spore. In some embodiments, the one or more microorganisms comprise Sporoschisma. In some embodiments, the one or more microorganisms comprise Sporothrix. In some embodiments, the one or more microorganisms comprise Sporotrichum. In some embodiments, the one or more microorganisms comprise Stachybotrys. In some embodiments, the one or more microorganisms comprise Stemphylium. In some embodiments, the one or more microorganisms comprise Syncephalastrum. In some embodiments, the one or more microorganisms comprise Thermononespore fusca DSM43793. In some embodiments, the one or more microorganisms comprise Torula, Trichocladium. In some embodiments, the one or more microorganisms comprise Trichoderma. In some embodiments, the one or more microorganisms comprise Trichophyton. In some embodiments, the one or more microorganisms comprise Trichothecium. In some embodiments, the one or more microorganisms comprise tritirachium, Ulocladium. In some embodiments, the one or more microorganisms comprise Verticillium. In some embodiments, the one or more microorganisms comprise Wallemia. In some embodiments, the one or more microorganisms comprises yeast.

### Methods of Producing SAPs and Intermediates of SAPs

In some embodiments, methods of preparing a SAP comprises the steps: (a) polymerizing aspartic acid monomers using a phosphoric acid (H₃PO₄) catalyst to form a polysuccinimide polymer; (b) crosslinking the polysuccinimide polymer in an organic solvent using one or more crosslinking agents to form a crosslinked polysuccinimide polymer; and (c) hydrolyzing the crosslinked polysuccinimide polymer using sodium carbonate (Na₂CO₃) to form a sodium salt form of crosslinked poly(aspartic acid) polymer. In some embodiments, the crosslinked polysuccinimide polymer is hydrolyzed using sodium hydroxide (NaOH) to form a sodium salt form of crosslinked poly(aspartic acid) polymer.

In an exemplary embodiment, a polysuccinimide (PSI) polymer (also sometimes referred to as polyanhydroaspartic acid or polyaspartimide), is formed during a thermal polycondensation of aspartic acid.

In some embodiments, methods of preparing a SAP comprises polymerizing of the aspartic acid monomers using the phosphoric acid (H₃PO₄) catalyst is performed at a temperature of from 150° C. to 300° C. In some embodiments, method of preparing a SAP comprise polymerizing of the aspartic acid monomers using the phosphoric acid (H₃PO₄) catalyst at a temperature of from 150° C. to 250° C. In some embodiments, preparing a SAP comprises the polymerizing of the aspartic acid monomers using the phosphoric acid (H₃PO₄) catalyst at a temperature of from 150° C. to 200° C. In some embodiments, the phosphoric acid (H₃PO₄) catalyst is reacted at a temperature of about 150° C., about 155° C., about 160° C., about 165° C., about 170° C., about 175° C., about 180° C., about 185° C., about 190° C., about 195° C., about 200° C., about 205° C., about 210° C., about 215° C., about 220° C., about 225° C., about 230° C., about 235° C., about 240° C., about 245° C., about 250° C., about 255° C., about 260° C., about 265° C., about 270° C., about 275° C., about 280° C., about 285° C., about 290° C., about 295° C., or about 300° C.

In some embodiments, methods of preparing a SAP comprises polymerizing of the aspartic acid monomers using the phosphoric acid (H₃PO₄) catalyst is performed at a temperature of from at least about 150° C. to at least about 300° C. In some embodiments, method of preparing a SAP comprise polymerizing of the aspartic acid monomers using the phosphoric acid (H₃PO₄) catalyst at a temperature of from at least about 150° C. to at least about 250° C. In some embodiments, preparing a SAP comprises the polymerizing of the aspartic acid monomers using the phosphoric acid (H₃PO₄) catalyst at a temperature of from at least about 150° C. to at least about 200° C. In some embodiments, the phosphoric acid (H₃PO₄) catalyst is reacted at a temperature of at least about 150° C., at least about 155° C., at least about 160° C., at least about 165° C., at least about 170° C., at least about 175° C., at least about 180° C., at least about 185° C., at least about 190° C., at least about 195° C., at least about 200° C., at least about 205° C., at least about 210° C., at least about 215° C., at least about 220° C., at least about 225° C., at least about 230° C., at least about 235° C., at least about 240° C., at least about 245° C., at least about 250° C., at least about 255° C., at least about 260° C., at least about 265° C., at least about 270° C., at least about 275° C., at least about 280° C., at least about 285° C., at least about 290° C., at least about 295° C., or at least about 300° C.

In some embodiments, methods of preparing a SAP comprise the steps: (a) polymerizing aspartic acid monomers using a phosphoric acid (H₃PO₄) catalyst to form a polysuccinimide polymer; (b) crosslinking the polysuccinimide polymer in an organic solvent using 1,8-diaminooctane as a crosslinking agent to form a crosslinked polysuccinimide polymer; and (c) hydrolyzing the crosslinked polysuccinimide polymer using sodium carbonate (Na₂CO₃) to form a sodium salt of crosslinked poly(aspartic acid) polymer.

In some embodiments, methods of preparing a SAP comprise the steps: (a) polymerizing aspartic acid monomers using a phosphoric acid (H₃PO₄) catalyst to form a polysuccinimide polymer; (b) crosslinking the polysuccinimide polymer in an organic solvent using 1,8-diaminooctane and cystamine as crosslinking agents to form a crosslinked polysuccinimide polymer; and (c) hydrolyzing the crosslinked polysuccinimide polymer using sodium carbonate (Na₂CO₃) to form a sodium salt of crosslinked poly(aspartic acid) polymer. In some embodiments, the organic solvent comprises dimethylsulfoxide (DMSO). In some embodiments, the organic solvent comprises diphenyl acetone (DPA). In some embodiments, the organic solvent comprises cyrene. In some embodiments, the SAP binds, adsorbs, absorbs, dissolves, sequesters, chelates, or any combination thereof, one or more fluids, a charged species, a biological molecule, an organic compound, or metal, or any combination thereof by at least 5%, to at least 200% or greater when compared to a super absorbent polymer prepared using a conventional method. In some embodiments, the SAP binds, adsorbs, absorbs, dissolves, sequesters, chelates, or any combination thereof, one or more fluids, a charged species, a biological molecule, an organic compound, or metal, or any combination thereof by at least about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 100%, about 105%, about 110%, about 115%, about 120%, about 125%, about 130%, about 135%, about 140%, about 145%, about 150%, about 155%, about 160%, about 165%, about 170%, about 175%, about 180%, about 185%, about 190%, about 195%, or about 200% of the SAP.

In some embodiments, methods of preparing a SAP comprise the steps: (a) polymerizing aspartic acid monomers using a phosphoric acid (H₃PO₄) catalyst to form a polysuccinimide polymer; (b) crosslinking the polysuccinimide polymer in an organic solvent using 1,8-diaminooctane and cystamine as crosslinking agents to form a crosslinked polysuccinimide polymer; and (c) hydrolyzing the crosslinked polysuccinimide polymer using sodium carbonate (Na₂CO₃) to form a sodium salt of crosslinked poly(aspartic acid) polymer. In some embodiments, the organic solvent comprises dimethylsulfoxide (DMSO). In some embodiments, the organic solvent comprises diphenyl acetone (DPA). In some embodiments, the organic solvent comprises cyrene. In some embodiments, the SAP binds, adsorbs, absorbs, dissolves, sequesters, chelates, or any combination thereof, one or more fluids, a charged species, a biological molecule, an organic compound, or metal, or any combination thereof by at least 5%, to at least 200% or greater when compared to a super absorbent polymer prepared using a conventional method. In some embodiments, the SAP binds, adsorbs, absorbs, dissolves, sequesters, chelates, or any combination thereof, one or more fluids, a charged species, a biological molecule, an organic compound, or metal, or any combination thereof by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, or at least about 200% of the SAP.

### Intermediates and Systems

In some embodiments, SAP intermediates comprise: (a) a polysuccinimide polymer; (b) a poly(aspartic acid) polymer or salt thereof, crosslinked with one or more crosslinking agents; (c) a phosphoric acid (H₃PO₄) catalyst; (d) an organic solvent; (e) sodium carbonate (Na₂CO₃); and (f) a salt of crosslinked poly(aspartic acid) polymer. In some embodiments, the salt of the crosslinked poly(aspartic acid) polymer is a sodium salt or a potassium salt. In some embodiments, the one or more crosslinking agents comprise any of the crosslinkers described herein. In some embodiments, the one or more crosslinking agents comprise at least one, at least two, at least three, at least four, or at least five crosslinkers. In some embodiments, the one or more crosslinking agents comprise two crosslinkers. In some embodiments, the one or more crosslinking agents comprise three crosslinkers. In some embodiments, the crosslinking agent is present at a concentration range of from 0.1 wt % to 10 wt %, from 1 wt % to 5 wt %, 2 wt % to 4 wt %, or 2.5 wt % of the SAP. In some embodiments, the crosslinking agents is present at a concentration range of from at least about 0.1 wt % to at least about 10 wt %, from at least about 1 wt % to at least about 5 wt %, from at least about 2 wt % to at least about 4 wt % of the SAP. In some embodiments, the organic solvent comprises dimethylsulfoxide (DMSO), diphenyl acetone (DPA), cyrene or a combination thereof.

In some embodiments, the one or more crosslinking agents comprises 1,8-diaminooctane. In some embodiments, the 1,8-diaminooctane is present at a concentration of from 0.1 wt % to 10 wt % of the SAP. In some embodiments, wherein the 1,8-diaminooctane is present at a concentration of from 1 wt % to 5 wt % of the SAP. In some embodiments, the 1,8-diaminooctane is present at a concentration of from 2 wt % to 4 wt % of the SAP. In some embodiments, the 1,8-diaminooctane is present at a concentration of 2.5 wt % of the SAP. In some embodiments, the 1,8-diaminooctane is present at a concentration of from at least about 0.1 wt % to at least about 10 wt % of the SAP. In some embodiments, wherein the 1,8-diaminooctane is present at a concentration of from at least about 1 wt % to at least about 5 wt % of the SAP. In some embodiments, the 1,8-diaminooctane is present at a concentration of from at least about 2 wt % to at least about 4 wt % of the SAP. In some embodiments, the 1,8-diaminooctane is present at a concentration of 2.5 wt % of the SAP. In some embodiments, the one or more crosslinking agents comprises cystamine. In some embodiments, the cystamine is present in a concentration of from 0.1 wt % to 10 wt % of the SAP. In some embodiments, the cystamine is present in a concentration of from 1 wt % to 5 wt % of the SAP. In some embodiments, the cystamine is present in a concentration of from 2 wt % to 4 wt % of the SAP. In some embodiments, the cystamine is present in a concentration of 2.5 wt % of the SAP. In some embodiments, the one or more crosslinking agents comprises cystamine. In some embodiments, the cystamine is present in a concentration of from at least about 0.1 wt % to at least about 10 wt % of the SAP. In some embodiments, the cystamine is present in a concentration of from at least about 1 wt % to at least about 5 wt % of the SAP. In some embodiments, the cystamine is present in a concentration of from at least about 2 wt % to at least about 4 wt % of the SAP. In some embodiments, the cystamine is present in a concentration of 2.5 wt % of the SAP. In some embodiments, the one or more crosslinking agents comprises 1,8-diaminooctane and cystamine. In some embodiments, the 1,8-diaminooctane is present in a concentration of 2.5 wt % of the SAP and the cystamine is present in a concentration of 2.5 wt % of the SAP. In some embodiments, the 1,8-diaminooctane is present in a concentration of 2.5 wt % of the SAP and the cystamine is present in a concentration of 2.5 wt % of the SAP. In some embodiments, the organic solvent comprises dimethylsulfoxide (DMSO). In some embodiments, the organic solvent comprises diphenyl acetone (DPA). In some embodiments, the organic solvent comprises cyrene.

In some embodiments, systems of preparing a SAP comprise: (a) aspartic acid monomers; (b) a phosphoric acid (H₃PO₄) catalyst; (c) a crosslinked polysuccinimide polymer; (d) an organic solvent; (e) one or more crosslinking agents; and (f) sodium carbonate (Na₂CO₃) base. In some embodiments, the one or more crosslinking agents comprises 1,8-diaminooctane. In some embodiments, the 1,8-diaminooctane is present at a concentration of from 0.1 wt % to 10 wt % of the SAP. In some embodiments, the 1,8-diaminooctane is present at a concentration of from 1 wt % to 5 wt % of the SAP. In some embodiments, the 1,8-diaminooctane is present at a concentration of from 2 wt % to 4 wt % of the SAP. In some embodiments, the 1,8-diaminooctane is present at a concentration of 2.5 wt % of the SAP. In some embodiments, the 1,8-diaminooctane is present at a concentration of from at least about 0.1 wt % to at least about 10 wt % of the SAP. In some embodiments, the 1,8-diaminooctane is present at a concentration of from at least about 1 wt % to at least about 5 wt % of the SAP. In some embodiments, the 1,8-diaminooctane is present at a concentration of from at least about 2 wt % to at least about 4 wt % of the SAP. In some embodiments, the 1,8-diaminooctane is present at a concentration of 2.5 wt % of the SAP. In some embodiments, the one or more crosslinking agents comprises cystamine. In some embodiments, the cystamine is present in a concentration of from 0.1 wt % to 10 wt % of the SAP. In some embodiments, the cystamine is present in a concentration of from 1 wt % to 5 wt % of the SAP. In some embodiments, the cystamine is present in a concentration of from 2 wt % to 4 wt % of the SAP. In some embodiments, the cystamine is present in a concentration of 2.5 wt % of the SAP. In some embodiments, the cystamine is present in a concentration of from at least about 0.1 wt % to at least about 10 wt % of the SAP. In some embodiments, the cystamine is present in a concentration of from at least about 1 wt % to at least about 5 wt % of the SAP. In some embodiments, the cystamine is present in a concentration of from at least about 2 wt % to at least about 4 wt % of the SAP. In some embodiments, the cystamine is present in a concentration of 2.5 wt % of the SAP. In some embodiments, the one or more crosslinking agents comprises 1,8-diaminooctane and cystamine. In some embodiments, the 1,8-diaminooctane is present in a concentration of 2.5 wt % of the SAP and the cystamine is present in a concentration of 2.5 wt % of the SAP. In some embodiments, the 1,8-diaminooctane is present in a concentration of 2.5 wt % of the SAP and the cystamine is present in a concentration of 2.5 wt % of the SAP. In some embodiments, the organic solvent comprises dimethylsulfoxide (DMSO). In some embodiments, the organic solvent comprises diphenyl acetone (DPA). In some embodiments, the organic solvent comprises cyrene.

### Articles of Manufacture and Methods of Use

Provided herein are articles of manufacture comprising a SAP of the present disclosure, wherein the articles of manufacture are substantially free of microplastics. As used herein, “substantially free” refers to a level that is less than 0.1 wt %, less than 0.01 wt %, or less than 0.001 wt % of a total weight of the SAP. The term “microplastics” as used herein comprises synthetic polymer microparticles that are solid polymers with maximum dimensions equal to or smaller than 5 mm, or length equal to or smaller than 15 mm in the case of fiber-like particles with a length-to-diameter ratio greater than three. The SAP of the present disclosure complies with European Union (EU) regulations under the Commission Regulation (EU) 2023/2055 (Annex XVII), Registration, Evaluation, Authorization and Restriction of Chemicals (REACH) regulation, as part of the EU's broader Zero Pollution Action Plan.

Provided herein are articles of manufacture comprising a SAP of the present disclosure, wherein the article of manufacture is an absorbent article. In some embodiments, an absorbent article comprises a SAP of the present disclosure, wherein the article of manufacture is a diaper, a sanitary pad, an incontinence product, a bandage, a wound dressing, a surgical pad, a pet pad, a bed pad, a meat pad, or an agricultural product. In some embodiments the article of manufacture comprising a SAP of the present disclosure is a diaper. In some embodiments the article of manufacture comprising a SAP of the present disclosure is a sanitary pad. In some embodiments the article of manufacture comprising a SAP of the present disclosure is an incontinence product. In some embodiments the article of manufacture comprising a SAP of the present disclosure is a bandage. In some embodiments the article of manufacture comprising a SAP of the present disclosure is a wound dressing. In some embodiments the article of manufacture comprising a SAP of the present disclosure is a surgical pad. In some embodiments the article of manufacture comprising a SAP of the present disclosure is a pet pad. In some embodiments the article of manufacture comprising a SAP of the present disclosure is a bed pad. In some embodiments the article of manufacture comprising a SAP of the present disclosure is a meat pad.

### Agricultural Products and Uses

In some embodiments the article of manufacture comprising a SAP of the present disclosure is an agricultural product. In some embodiments, the article of manufacture is used as a soil additive for water retention. In some embodiments, the article of manufacture is a desertification control (sand-fixing). In some embodiments, the article of manufacture is a seed or fertilizer coating.

In some embodiments the article of manufacture comprising a SAP of the present disclosure is used as an agricultural product. In some embodiments, the article of manufacture is used as a soil additive for water retention. In some embodiments, the article of manufacture is used as a desertification control (sand-fixing). In some embodiments, the article of manufacture is used as seed or fertilizer coatings.

### Hygiene Products and Uses

In some embodiments, the article of manufacture comprising a SAP of the present disclosure is a hygiene product In some embodiments, the article of manufacture is a disposable diaper. In some embodiments, the article of manufacture is an adult incontinence pad. In some embodiments, the article of manufacture is a sanitary napkin.

In some embodiments, the article of manufacture is used as a hygiene product. In some embodiments, the article of manufacture is used in disposable diapers. In some embodiments, the article of manufacture is used in adult incontinence pads. In some embodiments, the article of manufacture is used in sanitary napkins.

### Medical Products and Uses

In some embodiments, the article of manufacture comprising a SAP of the present disclosure is a medical or biomedical product. In some embodiments, the article of manufacture is a wound dressing. In some embodiments, the article of manufacture is a drug delivery hydrogel. In some embodiments, the article of manufacture is a tissue engineering scaffold. In some embodiments, the article of manufacture is a surgical pad. In some embodiments, the article of manufacture is a drug delivery system. In some embodiments, the article of manufacture is a tissue engineering product. In some embodiments, the article of manufacture is a surgical hygiene product.

In some embodiments, the article of manufacture is used as a medical or biomedical product. In some embodiments, the article of manufacture is used in wound dressings. In some embodiments, the article of manufacture is used as drug delivery hydrogels. In some embodiments, the article of manufacture is used in tissue engineering scaffolds. In some embodiments, the article of manufacture is used in wound dressings. In some embodiments, the article of manufacture is used in surgical pads. In some embodiments, the article of manufacture is used in drug delivery systems. In some embodiments, the article of manufacture is used in tissue engineering. In some embodiments, the article of manufacture is used in surgical hygiene products.

### Packaging Products and Use

In some embodiments, the article of manufacture comprising a SAP of the present disclosure is a packaging and/or food-absorbent article. In some embodiments, the article of manufacture is a food packaging pad (e.g., meat pad). In some embodiments, the article of manufacture is a moisture regulator in packaging. In some embodiments, the article of manufacture is construction packaging.

In some embodiments, the article of manufacture is used as a packaging and/or food-absorbent article. In some embodiments, the article of manufacture is used as pads in food packaging (e.g., meat trays). In some embodiments, the article of manufacture is used as moisture regulators in packaging. In some embodiments, the article of manufacture is used in construction packaging.

### Industrial Products and Uses

In some embodiments, the article of manufacture comprising a SAP of the present disclosure is an industrial product. In some embodiments, the article of manufacture is a construction material. In some embodiments, the article of manufacture is a water treatment product. In some embodiments, the article of manufacture is an oil spill treatment product. In some embodiments, the article of manufacture is used a spill absorbent product. In some embodiments, the article of manufacture is a wastewater treatment product. In some embodiments, the article of manufacture is used as a scale/corrosion inhibitor. In some embodiments, the article of manufacture is a flocculant for suspensions. In some embodiments, the article of manufacture is a heavy-metal chelating agent. In some embodiments, the article of manufacture is a mining and/or mineral processing product. In some embodiments, the article of manufacture is used as a dust control product. In some embodiments, the article of manufacture is a polymer spray on haul roads and ore piles. In some embodiments, the article of manufacture is used as a tailings management product. In some embodiments, the article of manufacture is used for tailings of slurries solidification product.

In some embodiments, the article of manufacture is used as an industrial product. In some embodiments, the article of manufacture is used in construction materials. In some embodiments, the article of manufacture is used in water treatment. In some embodiments, the article of manufacture is used in oil spill treatment. In some embodiments, the article of manufacture is used in spill absorbent kits. In some embodiments, the article of manufacture is used as a wastewater treatment product. In some embodiments, the article of manufacture is used as a scale/corrosion inhibitor in cooling systems. In some embodiments, the article of manufacture is used as a flocculant for suspensions. In some embodiments, the article of manufacture is used as a heavy-metal chelating agent. In some embodiments, the article of manufacture is a mining and/or mineral processing product. In some embodiments, the article of manufacture is used as a dust control product. In some embodiments, the article of manufacture is used as a polymer spray on haul roads and ore piles. In some embodiments, the article of manufacture is used as a tailings management product. In some embodiments, the article of manufacture is used for solidification of tailings slurries.

### Environmental Products and Uses

In some embodiments, the article of manufacture comprising a SAP of the present disclosure is an enhanced water recovery product. In some embodiments, the article of manufacture is an environmental remediation product. In some embodiments, the article of manufacture is an acid mine drainage (AMD) treatment product. In some embodiments, the article of manufacture is a heavy-metal cleanup product. In some embodiments, the article of manufacture is a soil stabilization product for mine site revegetation. In some embodiments, the article of manufacture is a cable water-blocking agent. In some embodiments, the article of manufacture is a water treatment product. In some embodiments, the article of manufacture is an oil spill treatment product.

In some embodiments, the article of manufacture is used for enhanced water recovery. In some embodiments, the article of manufacture is used as an environmental remediation product. In some embodiments, the article of manufacture is used in acid mine drainage (AMD) treatment. In some embodiments, the article of manufacture is used in heavy-metal cleanup. In some embodiments, the article of manufacture is used in soil stabilization for mine site revegetation. In some embodiments, the article of manufacture is used as a cable water-blocking agent. In some embodiments, the article of manufacture is used in water treatment. In some embodiments, the article of manufacture is used in oil spill treatment.

### EXAMPLES

### Example 1

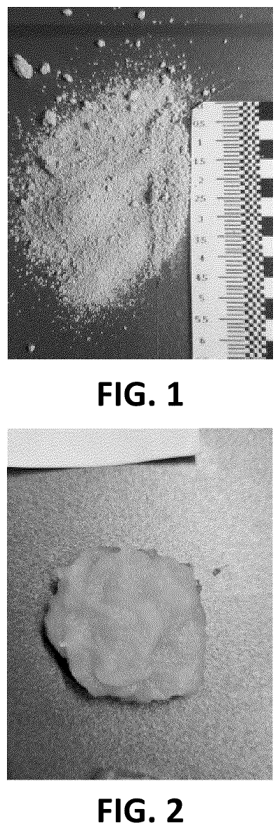

An exemplary SAP of the present disclosure (as shown in FIG. 1), was tested in a quickscreen test, based on ISO 20200:2024.

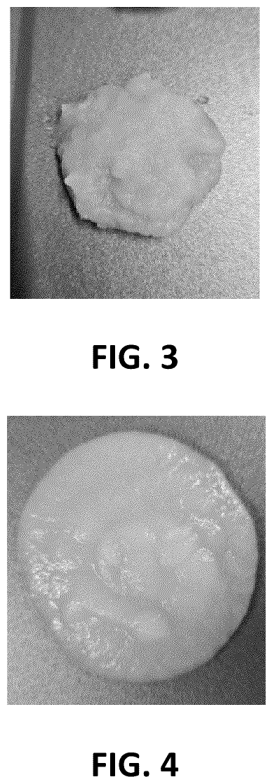

Three samples of the SAP were mixed with water (FIG. 2), water and an enzyme (FIG. 3), and saline and an enzyme (FIG. 4), respectively. All three sample, manifested as a hydrogen wafer, were shown to disintegrate by day 20 in the Quickscreen home compositing test, as per the method for ISO 20200:2024.

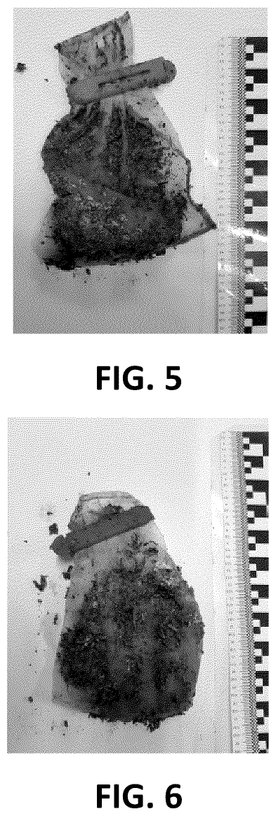

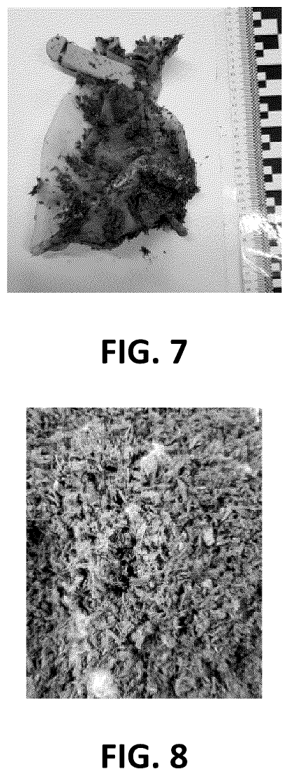

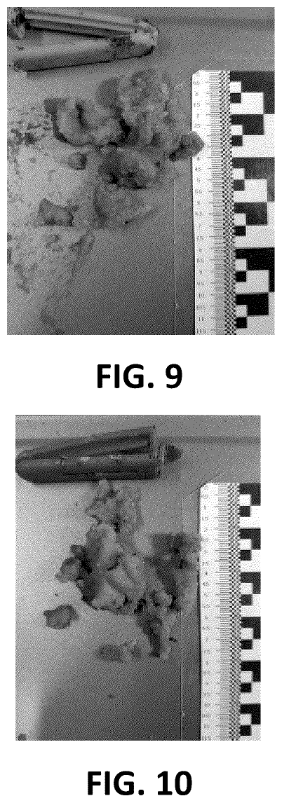

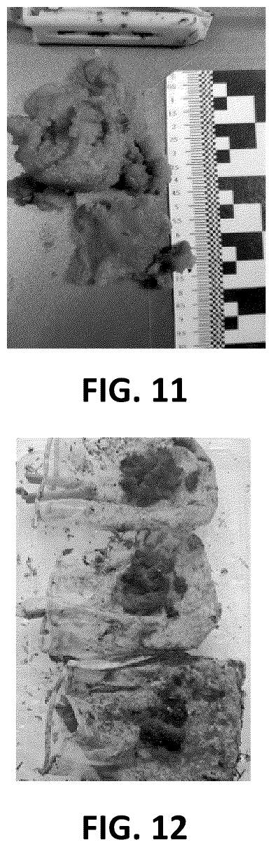

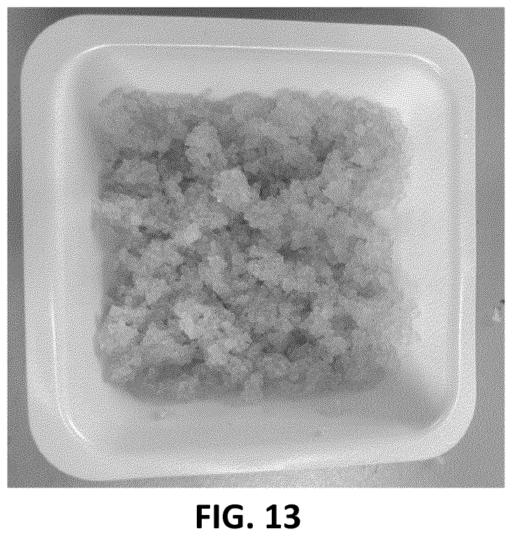

The experimental design and results are laid out in Table 1 below, and in the accompanying figures, as described above. FIGS. 1-12 shown that the test SAP of the present disclosure is prone to the abiotic and biotic effects of home composting.

TABLE 1

| Test requirement | Details | | |
|---|---|---|---|
| Test code number | AUT039 | | |
| Test piece description | Light-brown, powder, reconstituted into a hydrogel wafer | | |
| Test method | 20 days at 25° C. in home composting conditions (“Quicktest”), | | |
| | based on ISO 20200: 2024 | | |
| Test experimental | FIG. 2 water (4000% of the powder mass) and test polymer | | |
| design | FIG. 3 water (4000% of the powder mass), 0.8 mg/ml trypsin | | |
| | enzyme, and test polymer | | |
| | FIG. 4 saline (0.9% concentration), 2000% of the 0.9% saline | | |
| | by mass, and 0.8 mg/ml trypsin enzyme | | |
| Observations of | Observation | Positive Change | Negative or |
| test pieces - | | | no change |
| Control (FIG. 2) | Disintegrating | Yes (less than FIG. 4) | — |
| | Softening | Yes | — |
| | Color | Darkening | — |
| | Size and shape | Diminishing | — |
| | Odor | Malodor | — |
| Observations of | Observation | Positive Change | Negative or |
| test pieces - | | | no change |
| water and enzyme | Disintegrating | Yes (most of all) | — |
| (FIG. 3) | Softening | Yes | — |
| | Color | Darkening | — |
| | Size and shape | Diminishing (almost | — |
| | | gone) | |
| | Odor | Malodor | |
| Observations of | Observation | Positive Change | Negative or |
| test pieces - | | | no change |
| saline and enzyme | Disintegrating | Less (less then FIG. 3) | — |
| (FIG. 4) | Softening | Yes | — |
| | Color | Darkening | — |
| | Size and shape | Diminishing | — |
| | Odor | Malodor | — |

This example looked at the differences between water-, saline-, and saline and trypsin-containing hydrogels (FIG. 2, FIG. 3, and FIG. 4, respectively). Trypsin is an example of a proteolytic enzyme. The results shown in Table 1 and in the accompanying figures indicate that the control, containing only water (FIG. 2) had disintegrated considerably by Day 20, and is on track for meeting standards set by EN 13432 and EN 14995 specifications. The water and enzyme disintegration was significantly higher than the control. The saline experiment, with a lower solid content, looks similar in size to the control, but after drying the residue was minimal compared to the control. The disintegration order was [water/enzyme/polymer] less than [saline/enzyme/polymer] less than [control].

A lack of disintegration in the saline/enzyme/polymer iteration could be enhanced with a higher quantity of enzyme having an increased activity, due to the ionic strength.

The pH of the water/polymer sample was determined to be 8.2 (1:50), which is a typical range for trypsin enzyme. A high dilution due to the hydrophilic/hydrogel nature of the polymer, whereas at a lower dilution the pH will be higher.

Without wishing to be bound to theory, the inclusion of an enzyme in water- and saline-containing polymers of the present application appears to accelerate disintegration/breakdown of polymers described herein. It is also contemplated from the above observation that the hydrogel wafers show rapid signs of breakdown and following a positive breakdown trend of disintegrators previously tested in aerobic compost, digestion, or biodegradability tests.

### Example 2

Exemplary water absorption capacity sampling, testing and calculations were performed following ISO 17190-5: 2020 (E) with some variation, to account for the maximum amount of water absorption by the superabsorbent. Variables that were changed based on ISO17190-5: 2020 (E) include: (i) sampling amount of 0.018 g to 0.02 g of PASP sample was used for free swelling in deionized water; (ii) deionized water was used in place of saline for free swelling in water; and (iii) time of testing-samples was tested for 1 hr, 4 hr, 24 hr, and 48 hr in same deionized samples, blank were also tested in a similar way. A FSC maximum was found to be 1,200 g/g of deionized water.

AUL values were determined using EDANA NWSP 242.0.R2 (15) to determine absorption against weight.

### Example 3

An exemplary surface crosslinking method of the present disclosure is described below, using a direct coating of polyaspartic acid (PASP) with Ethylene Glycol Diglycidyl Ether (EGDGE) in the absence of acid:
(1) 1.5 wt % EGDGE was dissolved in 10 ml of solvent (15:85 ratio of water to acetone) to form an EGDGE solution.
(2) In a separate baking tray, 3 g of PASP was spread as a thin layer and the EGDGE solution was pipetted onto and evenly spread over the PASP layer to form a coated sample.
(3) The coated sample was left for 5 to 10 minutes at room temperature and then dried in a vacuum oven at 175° C. for 45 minutes to allow for surface crosslinking to occur.
(4) The oven-dried sample was then removed from the oven, crushed, and sieved to a selected size, e.g., >65%=300-600 μm.

### Example 4

An exemplary surface crosslinking method of the present disclosure is described below, with PASP acidification prior to PASP coating with EGDGE:

### Step 1: Polysuccinimide (PSI) Purification

(1) 40 g of unpurified PSI was placed in a beaker and dissolved in 400 ml of dimethyl sulfoxide (DMSO) at 45° C. for 2 hours and then left at room temperature overnight to form a PSI solution.
(2) The PSI solution was then centrifuged to remove any remaining insoluble particles and was then allowed to precipitate in 400 ml of deionized (DI) water. The precipitated PSI sample was then briefly mixed with a glass rod, left to stand for a few minutes, and then filtered.
(3) The filtrate was washed twice with DI water, once with methanol and then dried overnight in a vacuum oven at 60° C.
   Step 2: Cross-Linking with Diaminooctane (DAO)
(1) 20 mg of the purified PSI from Step 1 was dissolved in 200 ml of DMSO in a round bottom flask under magnetic stirring to form a PSI solution.
(2) 5 mol % DAO powder was added to the PSI solution and stirring was continued overnight to allow for cross-linking, to form a gel.
(3) The gel was broken up with a spatula and then a grinder and then filtered.
(4) The filtrate was mixed with 733 ml of DI water in a beaker to form a second solution.

### Step 3: Acid/Base Hydrolysis

(1) Hydrolysis of the second solution was carried out by adding a sodium hydroxide (NaOH) solution (5.93 g of NaOH in 200 ml of DI water) dropwise, with overhead stirring, to form a basic solution.
(2) The overhead stirring was continued for 2 hours to allow for complete hydrolysis, and the basicity of the basic solution was monitored using pH strips.
(3) 44.32 ml of a 1M hydrochloric acid (HCl) solution was added drop-wise to the basic solution while stirring (to convert 30% of the basic salt groups to acid groups) and the pH of the basic solution was periodically tested, showing the gradual change from the basic solution with a pH of about 12 to an acidic solution with a pH of about 4.5.
(4) The acidic solution was then allowed to precipitate in about 2 l of methanol, washed with methanol, and then filtered.
(5) The resultant filtrate was dried in a vacuum oven overnight at 50° C. and then crushed to form smaller crushed PASP particles.
   Step 4: Coating with EDGDE
(1) 1.5 wt % EGDGE was dissolved in 5 ml of solvent (15:85 ratio of water to acetone) to form an EGDGE solution.
(2) In a separate baking tray, 3 g of the crushed PASP particles from Step 3 was spread as a thin layer and the EGDGE solution was evenly spread over the layer to form a coated sample.
(3) The coated sample was then left for 5 to 10 minutes at room temperature and then dried in a vacuum oven at 175° C. for 45 minutes to allow for surface crosslinking to occur.
(4) The oven-dried sample was then removed from the oven, crushed, and sieved to a selected size, e.g., >65%=300-600 μm, as shown in FIG. 13 of the accompanying drawings.

### Example 5

An exemplary surface cross-linking method of the present disclosure is described below using direct coating of PASP with EGDGE and acetic acid:
(1) 1.5 wt % EGDGE was dissolved in 10 ml of solvent (15:85 ratio of water to acetone) and 20 mol wt % acetic acid.
(2) In a separate baking tray, 3 g of PASP was spread as a thin layer and the EGDGE solution was evenly spread over the layer to form a coated sample.
(3) The coated sample was left for 5 to 10 minutes at room temperature and then dried in a vacuum oven at 175° C. for 45 minutes to allow for surface crosslinking to occur.
(4) The oven-dried sample was then removed from the oven, crushed, and sieved to a selected size, e.g., >65%=300-600 μm.

### Example 6

Particle size distribution may be prepared in accordance with customer or end use requirements, e.g., a particle size range between 150 μm and 850 μm with the majority of the sample consisting of particles between 300 μm and 600 μm. In some instances, at a minimum, 65% of the sample mass is required to be within the size range of 300 μm to 600 μm, with a maximum of 19% being between 150 μm to 300 μm, and 15% being between 600 μm and 800 μm. At lab scale, the sample volume is ground using a mortar and pestle and sieved to assure size.

### Example 7

Colorimetry testing procedures for the WI values disclosed herein were determined using the following steps:

A colorimeter was turned on by pressing and holding a TEST button on the colorimeter and its black cap was removed in order to conduct a test.

The device was calibrated using a tool provided with the machine, which has one end for white calibration, and an opposite end for black calibration. A white calibration is carried out a few times. In order to select which calibration is being conducted, a dotted outline is located and the tool is move between the outline using up and down arrows.

Once calibrated, the colorimeter is used to measure a sample to get a reading, and the resultant values can be viewed and saved.

### Example 8

The following table 2 shows the Free Swell Capacity (FSC), Absorbance under Load (AUL) and centrifuge retention capacity (CRC) of exemplary superabsorbent polymers (SAPs) of the present application as compared to commercially available fossil fuel based SAPs and a sodium polyacrylate control.

TABLE 2

| | | Free Swell | Absorbance | Centrifuge |
|---|---|---|---|---|
| | | Capacity | Under Load | Retention |
| | SAP | (FSC) | (AUL) at 0.7 | Capacity |
| SAP Category | product | (g/g) | psi (g/g) | (g/g) |
| Exemplary SAPs | SAP 1 | 53 | 9 | 43 |
| of the present | SAP 2 | 38 | not tested | 25 |
| application | SAP 3 | not tested | not tested | 32 |
| | SAP 4 | 31 | 7 | 18 |
| | SAP 5 | 40 | 9.3 | 18 |
| Control fossil- | Sodium | 58 | 15.3 | 33 |
| based SAP | polyacrylate | | | |

As shown in the above table, SAP 1 of the present application showed a higher CRC compared to the control and commercially available SAPs.

### Example 9

A biodegradability test was carried out with a hydrated exemplary SAP (SAP 6) of the present application. The performance parameters, as determined using ISO methodology, of this exemplary SAP are shown in Table 3, below.

TABLE 3

| Parameter | SAP 6 |
|---|---|
| Water Absorption (g/g) | 274 |
| Free Swell Capacity (FSC) (g/g) (ISO 17190-5) | 56 |
| Centrifuge Retention Capacity (g/g) (ISO/DIS 17190-6) | 36 |
| Absorbance Under Load (AUL) at 0.7 psi (g/g) (ISO 17190-8) | 9 |

The test showed that the exemplary SAP 6 were clearly disintegrating by day 20 in home composting and by day 10 in industrial-scale composting. Both results were determined using ISO 20200:2023. The hydrated SAP showed rapid signs of breakdown and followed a positive breakdown trend of disintegrators which have previously been tested in aerobic compost, digestion, or biodegradability tests. The carbon footprint of this SAP 6 was 1.70 kg CO₂/kg SAP.

The speed of the breakdown of this exemplary SAP of the present application is very high and would therefore meet the 84-day threshold provided for in ISO 20200:2023. The material is susceptible to biotic and abiotic effects. Surrounding compost of the SAP under these conditions do not exhibit ill-effects and appear to benefit from the mycelial growth and cohesion of the tested SAP. At a very high moisture content, the SAP appears to be soluble, which is uncommon in industrial composting.

### Example 10

A 48-hour 2 application primary irritation patch test was conducted on exemplary SAP material of the present disclosure in a panel of twenty-two (22) healthy participants. All the participants have sensitive skin and were tested for approximately 23 hours each, over 3 days. Skin reactions were assessed on after the second and final day of the assessment. The test materials were evaluated was shown to be well tolerated under exaggerated conditions of the study, and produced irritation levels that were lower than a positive control and comparable to a negative control, i.e., no palpable skin irritations were observed for the test material.

The purpose of the study was to assess the potential of a SAP test material to elicit human skin irritation by repetitive topical application. The study provides data on the performance of the t SAP test materials under the conditions of the study (single-center, within-subject comparison, assessor blinded, randomized).

The administration route was a topical, occlusive patch. SAP test material, sodium lauryl sulfate (SLS) as a positive control, and sterile water as negative control were applied to the patch prior to application to each subject, time of preparation and application to the subject was recorded to confirm preparation and application times. The patch containing the test material was applied to either the lateral surface of the upper arms, midway between the shoulder and the elbow, or to the lower back (between the waistline and the lower edge of the shoulder blade) on either side of the spine. Skin markers were used to allow exact relocation to the test areas at subsequent applications. To assure good adhesion, patches may have been reinforced with additional tape. The test material was applied to the patch as follows: 0.2 g of granules were placed on a webril pad before 0.2 ml of saline was added prior to patch application. The patch was adhered to the skin using an occlusive tape. For the control material approximately 0.2 ml was applied to webril pads placed on to an occlusive tape. Approximately 23 hours after the first patch application, the subjects removed their patch and were advised to wipe (with water) the test sites to remove any residual test material. The test sites were assessed approximately 1 hour later by a trained skin assessor following a scoring system: 0-no visible reaction; 1-2-slight redness; 3-4-moderate redness; 5-6-strong reacting, distinct redness, possibly some oedema; and 7-8-very strong reaction with blistering.

After assessment of reactions, an identical patch scheme was reapplied to the same area for a further period of approximately 23 hours. The test materials were reapplied to the same site as before following assessment. However, a score of 5 or more for Erythema or Dryness as adjudged by the assessor was considered sufficiently severe to discontinue the testing of a material at that site. Similarly, any palpable score (oedema, vesicles and papules) associated with any erythematous score was also considered sufficiently severe to discontinue the testing of a material at that site. The negative control was applied neat, the test material was applied at a concentration of 100.0%. The positive control, sodium lauryl sulfate (SLS) was prepared and applied as a 0.3% w/v solution in sterile water.

The exemplary SAP material of the present disclosure was found to be dermatologist approved.

### Example 11

Test pieces of exemplary SAPs of the present disclosure were allowed to decompose over a 21 day period and then subjected to an ecotoxicological screen to test for the presence and levels of toxins, if present in the decomposed test pieces.

The metal content of the test pieces were found to comply with British Standards Institution PAS 100 standards, which is an industry standard for composts, and with U.S. and EU metal content regulations for the amounts permitted in soils. Although there were levels of detectable metals, those present in the test pieces represent background metal content that occurs in everyday compost that is manufactured for commercial use, the levels detected did not represent any cause for concern that the test pieces were a contributing source of metal contamination.

Trace detections of polycyclic aromatic hydrocarbons and chloro-meta-cresol (4-chloro-2-methylphenol) were also detected in both a control compost and the composts of the test pieces. Extractable petroleum hydrocarbons that are often present as mineral oil contamination of municipal composts were also detected at levels that are significantly lower than levels that would be of any concern.

### Test Piece Preparation

The test pieces were ground up (or cut up) to allow all the fragments to pass through a 2 mm sieve. 100 g of ground sample was then added to a mature compost mix that was specified by test method ISO 20200:2015. The ratio of test pieces to compost was 100 g (dry weight) of test pieces to 454 g (dry weight) of compost (1:4.6 dilution m/m dry).

A thermophilic test, run according to ISO 20200 was followed and the test ended after 21 days. The compost was then analyzed as noted below, and the results are reported below as found in the compost at the test piece to compost dilution given above.

### Metals

The measurement of metals examines the number of metals that are present in the compost. Many metals can be toxic to the soil environment at high concentrations. As an insoluble metal the metal cannot leach out of the soil and could result in a metal build-up of the soil. At high metals levels a temporary solubility, e.g., during acidic pH exposure, the metal will easily be absorbed by fauna and flora, and if at toxic levels, could result in negative environmental impacts. Levels of the following metals were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: arsenic (As), antimony (Sb), barium (Ba), beryllium (Be), cadmium (Cd), chromium (Cr), cobalt (Co), copper (Cu), mercury (Hg), lead (Pb), molybdenum (Mo), nickel (Ni), selenium (Se), tin (Sn), vanadium (V), and zinc (Zn).

### Volatile Organic Hydrocarbons (VOH)

Compounds present in compost (that have boiling points and are organic hydrocarbons in nature) can enter the soil and present themselves to humans or fauna. These compounds themselves can be toxic in short term exposure or can present health issues to animals/humans through long-term exposure. Levels of the following volatile organic hydrocarbons were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: benzene, ethylbenzene, toluene, o-xylene, m,p-xylene, xylenes, styrene, 1,2,4-trimethylbenzene, 1,3,5-trimethylbenzene, n-propylbenzene, isopropylbenzene, n-butylbenzene, sec-butylbenzene, tert-butylbenzene, p-isopropyltoluene, o/p-chloronitrobenzene, m-chloronitrobenzene, monochloronitrobenzenes, 2,3+3,4-dichloronitrobenzene, 2,4-dichloronitrobenzene, 2,5-dichloronitrobenzene, and 3,5-dichloronitrobenzene, dichloronitrobenzenes.

### Phenols

Some phenols are corrosive to skin and irritating in mucous membranes through protein disruption. Long term exposure to some phenolic compounds can cause kidney damage. Some cancer links have been attributed to certain phenolic substances. Acute toxicity to selected phenols is also present in phenolics that are easily absorbed through skin. Some phenolics can cause embryo development issues. Levels of the following phenols were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: phenol, o-cresol, m-cresol, p-cresol, cresol, 2,4-dimethylphenol, 2,5-dimethylphenol, 2,6-dimethylphenol, 3,4-dimethylphenol, o-ethylphenol, m-ethylphenol, thymol, and 2,3/3,5-dimethylphenol+4-ethylphenol.

### Polycyclic Aromatic Hydrocarbons (PAH)

Selected polycyclic aromatic hydrocarbons (PAHs) are cancerous, implicated in cardiovascular disease, and in embryo/foetus development (i.e., reprotoxic). PAHs can affect the DNA of organisms through mutational influence. The mutations often give rise to enzyme dysfunction. Levels of the following polycyclic hydrocarbons were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: naphthalene, acenaphthylene, acenaphthene, fluorene, phenanthrene, anthracene, fluoranthene, pyrene, benzo (a) anthracene, chrysene, benzo (b) fluoranthene, benzo (k) fluoranthene, benzo (a) pyrene, dibenzo (ah) anthracene, benzo (ghi) perylene, indeno (123cd) pyrene, PAH 10 VROM, and PAH 16 EPA.

### Volatile Halogenated Hydrocarbons (VHC)

Many haloalkanes (volatile halogenated hydrocarbons, VHCs) have been shown to have carcinogenic abilities, which when present in soil poses human and animal risks. VHCs can affect the DNA of organisms through mutational influence. The mutations often give rise to enzyme dysfunction. Levels of the following volatile halogenated hydrocarbons were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: tetrachloromethane; 1,2-dichloroethane; 1,1,1-trichloroethane; 1,1,2-trichloroethane; trichloroethanes; 1,1,1,2-tetrachloroethane; 1,1,2,2-tetrachloroethane; tetrachloroethanes; trichloroethene; tetrachloroethene; 1,2-dichloropropane; 1,3-dichloropropane; 1,2,3-trichloropropane; 1,1-dichloropropene; cis-1,3-dichloropropene; trans-1,3-dichloropropene; 1,3-dichloropropenes; dibromomethane; 1,2-dibromoethane; tribromomethane; bromodichloromethane; dibromochloromethane; 1,2-dibromo-3-chloropropane; and bromobenzene.

### Chlorobenzenes

Chlorobenzenes are harmful to humans and animals through inhalation or skin absorption. Through acute toxicity effects, organisms will be harmed at low or moderate levels. Chlorobenzenes will leach out of soil and will enter aquatic systems where they persist. In aquatic systems organisms are affected through acute toxic exposure. Levels of the following chlorobenzenes were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: monochlorobenzene; 1,3-dichlorobenzene; 1,4-dichlorobenzene; dichlorobenzenes; 1,2,3-trichlorobenzene; 1,2,4-trichlorobenzene; 1,3,5-trichlorobenzene; trichlorobenzenens; 1,2,3,4-tetrachlorobenzene; 1245&1235-tetrachlorobenzene; tetrachlorobenzenes; pentachlorobenzene; and hexachlorobenzene.

### Chlorophenols

Present in many biocides, especially fungicides the chlorinated phenols are sometimes present in soil or through treated materials that are composted. Levels of the following chlorophenols were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: o-chlorophenol; m-chlorophenol; p-chlorophenol; monochlorophenols; 2,3-dichlorophenol; 2,4/2,5-dichlorophenol; 2,6-dichlorophenol; 3,4-dichlorophenol; 3,5-dichlorophenol; dichlorophenols; 2,3,4-trichlorophenol; 2,3,5-trichlorophenol; 2,3,6-trichlorophenol; 2,4,5-trichlorophenol; 2,4,6-trichlorophenol; 3,4,5-trichlorophenol; trichlorophenols; 2,3,4,5-tetrachlorophenol; 2,3,4,6/2,3,5,6-tetrachlorophenol; tetrachlorophenols; pentachlorophenol; and 4-chloro-3-methylphenol.

### Polychlorinated Biphenyl (PCB)

PCBs are bioaccumulative, i.e., they are retained in body tissues and concentrate over time. Eventually PCB in bodily tissues reach a toxicity threshold that will harm the organism. Many of the PCBs are carcinogenic and the harm they do to organisms is through cancer. Levels of the following polychlorinated biphenyls were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: PCB 28; PCB 52; PCB 101; PCB 118; PCB 138; PCB 153; PCB 180; PCB (6); and PCB (7).

### Miscellaneous Chlorinated Hydrocarbons

The miscellaneous chlorinated hydrocarbons listed below are harmful if inhaled and have long lasting very toxic effects to environmental organisms. Further these compounds can cause reproductive harm and are flammable. Levels of the following chlorinated hydrocarbons were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: 2-chlorotoluene; 4-chlorotoluene; chlorotoluenes; and 1-chloronaphthalene.

### Organic Chlorinated Pesticides

Organochlorine pesticides are persistent in the environment. They can also bioaccumulate due to their lipophilic nature. Inside organisms their effects are very toxic. These very toxic effects include neurotoxic, reproductive, genotoxic, and tumerogenic effects. Levels of the following organic chlorinated pesticides were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: 4,4-DDE; 2,4-DDE; 4,4-DDT; 4,4-DDD+2,4-DDT; 2,4-DDD; DDT/DDE/DDD; aldrin; dieldrin; endrin; drins; alpha-HCH; beta-HCH; gamma-HCH; delta-HCH; 4 HCH-compounds; alfa-endosulfan; alfa-endosulfansulphate; alfa-chlordan; y-chlordan; chlordans; heptachlor; heptachloroepoxide; hexachlorobutadiene; isodrin; telodrin; and tedion.

### Phosphor Pesticides

Organophosphates act directly on acetylcholinesterase, an enzyme vital for healthy nervous system functioning. The nervous systems of humans, insects, and other animals are affected by organophosphates. Organophosphates can degrade in soils, but their acute effects are of concern. Levels of the following phosphor pesticides were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: azinphos-ethyl; azinphos-methyl; bromophos-ethyl; bromophos-methyl; chloropyriphos-ethyl; chloropyriphos-methyl; cumaphos; demeton-S/demeton-O-ethyl; diazinone; disulphotone; fenitrothion; fenthion; malathion; parathion-ethyl; parathion-methyl; pyrazophos; and triazophos.

### Nitrogen Pesticides

Many of the nitrogen pesticides, through their extensive use as herbicides, are present in soil and composts. The effects they have on the ecosystem is generally through reprotoxic effects. These reprotoxic effects are generally endocrine disruption which can include teratogenic effects (emasculinisation of fish and other organisms). General endocrine disruption will include mimicry of sex hormones. Levels of the following nitrogen pesticides were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: ametryn; atrazine; cyanazine; desmetryn; prometryn; propazine; simazine; terbutylazine; and terbutryn.

### Miscellaneous Pesticides

The pesticides listed below are often insecticides that target the nervous systems of insects. It has been discovered that many of them will leach into aquatic systems and influence very toxic effects on the organisms in that aquatic system. Some of the pesticides listed below can be irritating on contact with animal skin. Levels of the following pesticides were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: bifenthrin; cypermethrin A, B, C and D; deltamethrin; permethrin (A+B); propachlor; and trifluralin.

### Miscellaneous Organic Compounds

The compounds listed below are mildly toxic but can form intermediates or products that are listed elsewhere in this ecotoxicology screen. Nitrobenzene is highly toxic that can be easily absorbed through the skin and will directly affect the nervous system. Dibenzofuran has long lasting effects on aquatic systems. Levels of the following organic compounds were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: biphenyl; nitrobenzene; and dibenzofuran.

### Phthalate Esters

All the phthalate esters listed below, through their extensive use as plasticizers are present in contaminated soil and composts. They are also present in aquatic systems through accidental or on-purpose release. The effects they have on the ecosystem is generally through reprotoxic effects. These reprotoxic effects are generally endocrine disruption which can include teratogenic effects. Levels of the following phthalate esters were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: dimethylphthalate; diethylphthalate; diisobutylphthalate; di-n-butylphthalate; butylbenzylphthalate; bisethylhexylphthalate (DEHP); di-n-octylphthalate; and phthalates.

### Petroleum Hydrocarbons (PH)

Organisms encountering a high total petroleum hydrocarbon (TPH) exposure will absorb the Extractable PH (EPH), through the skin and the chemicals will concentrate in the cell. The toxic effects of the EPH are then through narcosis, i.e., the accumulation of foreign chemistry in the cell that will result in toxic effects. Acute as well as chronic toxicity can result, depending on the nature of the PH absorbed. Levels of the following petroleum hydrocarbons were measured in the exemplary test pieces of the SAPs disclosed herein using either gas chromatography-mass spectrometry (GC-MS), gravimetry, inductively coupled plasma-mass spectrometry (ICP-MS), or mass spectrometry-flame ionization detection (MS-FID) analysis, and were found to be at levels of no concern (i.e., below detection levels) or at very low detections levels: EPH (C10-C12); EPH (C12-C16); EPH (C16-C21); EPH (C21-C30); EPH (C30-C35); EPH (C35-C40); and EPH Sum (C10-C40).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EMBODIMENTS

### Embodiment 1

A superabsorbent polymer, comprising a poly(aspartic acid) polymer crosslinked with 1,8-diaminooctane, wherein the superabsorbent polymer has a water absorption capacity of from 10 g/g to 600 g/g, wherein the superabsorbent polymer has a saline absorption of from 10 g/g to 100 g/g, wherein the superabsorbent polymer has an absorption under load (AUL) of from 1 g/g to 100 g/g under a load of 0.7 psi, wherein the superabsorbent polymer is degraded upon hydrolysis at a temperature of 15° C. or higher in 20 days when measured by ISO 20200:2024 or a comparable assay.

### Embodiment 2

A superabsorbent polymer, comprising a poly(aspartic acid) (PASP) polymer crosslinked with 1,8-diaminooctane and cystamine; wherein the superabsorbent polymer has a water absorption capacity of from 10 g/g to 600 g/g, and wherein the superabsorbent polymer is degraded upon hydrolysis at a temperature of 15° C. in 20 days when measured by ISO 20200:2024 or a comparable assay.

### Embodiment 3

The superabsorbent polymer of embodiments 1 or 2, comprising a free swell capacity (FSC) of from 20 g/g to 100 g/g under a 0.9% saline solution.

### Embodiment 4

The superabsorbent polymer of any one of embodiments 1-3, comprising a centrifuge retention capacity (CRC) of from 10 g/g to 50 g/g.

### Embodiment 5

The superabsorbent polymer of any one of embodiments 1-4, wherein the superabsorbent polymer comprises 50% or more biobased carbons.

### Embodiment 6

The superabsorbent polymer of any one of embodiments 1-5, wherein the 1,8-diaminooctane is present at a concentration of from 0.1 wt % to 15 wt % of the superabsorbent polymer.

### Embodiment 7

The superabsorbent polymer of any one of embodiments 2-6, wherein the cystamine is present at a concentration of from 0.1 wt % to 10 wt % of the superabsorbent polymer.

### Embodiment 8

The superabsorbent polymer of any one of embodiments 1-7, wherein the superabsorbent polymer has a whiteness index of L* in a range of from 95 to 100, a* in a range of from −1.5 to −1.1 and b* in a range of from 4.2 to 4.7.

### Embodiment 9

The superabsorbent polymer of any one of embodiments 1-8, further comprising a surface crosslinker selected from an epoxy; ethylene glycol diglycidyl ether (EGDGE); poly(ethylene glycol) diglycidyl ether (PEGDGE); ethylene glycol; diethyleneglycol diglycidyl ether; 1,4-butanediol diglycidyl ether; glycerol polyglycidyl ether; sorbitol polyglycidyl ether; 2-oxazolidinone; N-(2-hydroxy ethyl)-2 oxazolidinone (HEONON); ethylene carbonate; 1, 4 butane diol; glycerol; ethylene diamine; and 1, 4 butane diamine.

### Embodiment 10

The superabsorbent polymer of any one of embodiments 1-9, wherein the superabsorbent polymer is hydrolyzed on exposure to an enzymatic hydrolysis or chemical hydrolysis.

### Embodiment 11

The superabsorbent polymer of embodiment 10, wherein the enzymatic hydrolysis occurs upon contact of the superabsorbent polymer with one or more enzymes or one or more microorganisms.

### Embodiment 12

The superabsorbent polymer of embodiment 11, wherein the one or more enzymes comprises one or more lipolytic enzymes, one or more proteolytic enzymes, one or more amylolytic enzymes, one or more cellulolytic enzymes, or any combination thereof.

### Embodiment 13

The superabsorbent polymer of embodiment 11, wherein the one or more microorganisms comprises a bacterium or a yeast.

### Embodiment 14

The superabsorbent polymer of any one of embodiments 1-9, wherein the superabsorbent polymer is degradable upon exposure to solar radiation.

### Embodiment 15

The superabsorbent polymer of any one of embodiments 1-9, wherein the superabsorbent polymer degrades upon contact with an effective amount of environmental moisture.

### Embodiment 16

A biodegradable poly(aspartic acid) hydrogel, comprising a superabsorbent biopolymer comprising a poly(aspartic acid) polymer crosslinked with 1,8-diaminooctane and/or cystamine; and one or more fluids, a charged species, a biological molecule, an organic compound, a metal, or combinations thereof; wherein the superabsorbent polymer has a water absorption capacity of from 10 g/g to 600 g/g, and wherein the biodegradable poly(aspartic acid) hydrogel is degraded upon hydrolysis at a temperature of 15° C. in 20 days when measured by ISO 20200:2024 or a comparable assay.

### Embodiment 17

The biodegradable poly(aspartic acid) hydrogel of embodiment 16, wherein the one or more fluids comprises water, hydrocarbon, oil, alcohol, an aqueous solution, a non-aqueous solution, an ionic solution, a biological fluid, a gas, a waste-water, a fracking fluid, or any combination thereof.

### Embodiment 18

An article of manufacture comprising a superabsorbent polymer comprising a poly(aspartic acid) polymer crosslinked with 1,8-diaminooctane and cystamine, (a) wherein the superabsorbent polymer has a water absorption capacity of from 10 g/g to 600 g/g, (b) wherein the superabsorbent polymer is degraded upon hydrolysis at a temperature of 15° C. in 20 days when measured by ISO 20200:2024 or a comparable assay; and (c) wherein the article of manufacture is a diaper, a sanitary pad, an incontinence product, a bandage, a wound dressing, a surgical pad, a pet pad, a bed pad, or a meat pad.

### Embodiment 19

A superabsorbent polymer, comprising a crosslinked poly(aspartic acid) polymer; wherein the superabsorbent polymer has: a whiteness index of L* in a range of from 97 to 100, a* in a range of from −1.4 to −1.2 and b* in a range of from 4.3 to 4.6; a water absorption capacity of at least 500 g/g; and a free swell capacity (FSC) in a range of from 30 g/g to 80 g/g.

### Embodiment 20

The superabsorbent polymer of embodiment 19, wherein the superabsorbent polymer has a whiteness index with L* in a range of from 99 to 100, a* in a range of from −1.4 to −1.3 and b* in a range of from 4.4 to 4.5.

### Embodiment 21

The superabsorbent polymer of embodiment 19 or 20, wherein the superabsorbent polymer has a whiteness index of L*=95.5, a*=1.2 and b*=4.5.

### Embodiment 22

The superabsorbent polymer of any one of embodiments 19-21, wherein the superabsorbent polymer has a water absorption capacity of from 900 g/g to 1400 g/g.

### Embodiment 23

The superabsorbent polymer of any one of embodiments 19-22, wherein the superabsorbent polymer has a water absorption capacity of from 1000 g/g to 1100 g/g.

### Embodiment 24

The superabsorbent polymer of any one of embodiments 19-23, comprising a free swell capacity (FSC) of from 40 g/g to 70 g/g under a 0.9% saline solution.

### Embodiment 25

The superabsorbent polymer of any one of embodiments 19-24, comprising a FSC of from 50 g/g to 60 g/g under a 0.9% saline solution.

### Embodiment 26

The superabsorbent polymer of any one of embodiments 19-25, comprising a centrifuge retention capacity (CRC) of from 30 g/g to 70 g/g.

### Embodiment 27

The superabsorbent polymer of any one of embodiments 19-26, comprising a CRC of from 40 g/g to 70 g/g.

### Embodiment 28

The superabsorbent polymer of any one of embodiments 19-27, comprising a CRC of from 60 g/g to 70 g/g.

### Embodiment 29

The superabsorbent polymer of any one of embodiments 19-28, comprising an absorbance under load (AUL) of from 8 g/g to 20 g/g under a load of 0.7 psi.

### Embodiment 30

The superabsorbent polymer of any one of embodiments 19-29, comprising an AUL of from 10 g/g to 18 g/g under a load of 0.7 psi.

### Embodiment 31

The superabsorbent polymer of any one of embodiments 19-30, wherein the crosslinked poly(aspartic acid) polymer is crosslinked with 1,8-diaminooctane.

### Embodiment 32

The superabsorbent polymer of embodiment 31, wherein the 1,8-diaminooctane is present at a concentration of from 0.1 wt % to 15 wt % of the superabsorbent polymer.

### Embodiment 33

The superabsorbent polymer of embodiments 31 or 32, wherein the 1,8-diaminooctane is present at a concentration of from 1 wt % to 10 wt % of the superabsorbent polymer.

### Embodiment 34

The superabsorbent polymer of any one of embodiments 31-33, wherein the 1,8-diaminooctane is present at a concentration of from 2 wt % to 5 wt % of the superabsorbent polymer.

### Embodiment 35

The superabsorbent polymer of any one of embodiments 31-34, wherein the 1,8-diaminooctane is present at a concentration of 2.5 wt % of the superabsorbent polymer.

### Embodiment 36

The superabsorbent polymer of any one of embodiments 19-30, wherein the crosslinked poly(aspartic acid) polymer is crosslinked with cystamine.

### Embodiment 37

The superabsorbent polymer of embodiment 36, wherein the cystamine is present at a concentration of from 0.1 wt % to 10 wt % of the superabsorbent polymer.

### Embodiment 38

The superabsorbent polymer of embodiment 36 or 37, wherein the cystamine is present at a concentration of from 1 wt % to 5 wt % of the superabsorbent polymer.

### Embodiment 39

The superabsorbent polymer of any one of embodiments 36-38, wherein the cystamine is present at a concentration of from 2 wt % to 4 wt % of the superabsorbent polymer.

### Embodiment 40

The superabsorbent polymer of any one of embodiments 36-39, wherein the cystamine is present at a concentration of 2.5 wt % of the superabsorbent polymer.

### Embodiment 41

The superabsorbent polymer of any one of embodiments 19-40, further comprising a surface crosslinker.

### Embodiment 42

The superabsorbent polymer of embodiment 41, wherein the surface crosslinker is an epoxy; ethylene glycol diglycidyl ether (EGDGE); poly(ethylene glycol) diglycidyl ether (PEGDGE); ethylene glycol; diethyleneglycol diglycidyl ether; 1,4-butanediol diglycidyl ether; glycerol polyglycidyl ether; sorbitol polyglycidyl ether; 2-oxazolidinone; N-(2-hydroxy ethyl)-2 oxazolidinone (HEONON); ethylene carbonate; 1, 4 butane diol; glycerol; ethylene diamine; or 1, 4 butane diamine.

### Embodiment 43

The superabsorbent polymer of embodiment 41 or 42, wherein the surface crosslinker is EGDGE or PEGDGE.

### Embodiment 44

The superabsorbent polymer of any one of embodiments 19-43, wherein the superabsorbent polymer is hydrolyzed on exposure to an enzymatic hydrolysis or a chemical hydrolysis.

### Embodiment 45

The superabsorbent polymer of embodiment 44, wherein the enzymatic hydrolysis occurs upon contact of the superabsorbent polymer with one or more enzymes or one or more microorganisms.

### Embodiment 46

The superabsorbent polymer of embodiment 45, wherein the one or more enzymes comprises one or more lipolytic enzymes, one or more proteolytic enzymes, one or more amylolytic enzymes, one or more cellulolytic enzymes, or any combination thereof.

### Embodiment 47

The superabsorbent polymer of embodiment 46, wherein the one or more lipolytic enzymes comprises one or more carboxylesterases and/or lipases.

### Embodiment 48

The superabsorbent polymer of embodiment 46, wherein the one or more proteolytic enzymes comprises one or more serine proteases, cysteine proteases, threonine proteases, aspartic proteases, metalloproteases, glutamic proteases, or any combination thereof.

### Embodiment 49

The superabsorbent polymer of embodiment 48, wherein the one or more serine proteases comprises one or more trypsins, chymotrypsins, thrombins, subtilisins, or any combination thereof.

### Embodiment 50

The superabsorbent polymer of embodiment 46, wherein the one or more amylolytic enzymes comprise one or more α-amylases, β-amylases, glucoamylases, or any combination thereof.

### Embodiment 51

The superabsorbent polymer of embodiment 45, wherein the one or more microorganisms comprises a bacterium or a yeast.

### Embodiment 52

The superabsorbent polymer of embodiment 45 or 51, wherein the one or more microorganisms comprises psychrophiles, mesophiles, thermophiles, actinomycetes, saprophytes, Absidia, Acremonium, Alternaria, amerospore, Arthrinium, ascospore, Aspergillus, Aspergillus caesiellus, Aspergillus candidus, Aspergillus carneus, Aspergillus clavatus, Aspergillus deflectus, Aspergillus flavus, Aspergillus fumigatus, Aspergillus glaucus, Aspergillus nidulans, aspergillus ochraceus, Aspergillus oryzae, Aspergillus parasiticus, Aspergillus penicilloides, aspergillus restrictus, Aspergillus sydowii, Aspergillus terreus, Aspergillus ustus, Aspergillus versicolor, Aspergillus/Penicillium-like, Aureobasidium, basidiomycetes, basidiospore, Bipolaris, Blastomyces, B. borstelensis, Botrytis, Candida, Cephalosporium, Chaetomium, Cladosporium, Cladosporium fulvum, Cladosporium herbarum, Cladosporium macrocarpum, Cladosporium sphaerospermum, conidia, conidium, conidobolus, Cryptococcus neoformans, cryptostroma corticale, Cunninghamella, Curvularia, dreschlera, Epicoccum, Epidermophyton, Fungus, Fusarium, Fusarium solani, Geotrichum, Gliocladium, Helicomyces, Helminthosporium, Histoplasma, humicula, Hyaline mycelia, Memnoniella, Microsporum, mold, Monilia, Mucor, mycelium, myxomycetes, Nigrospora, Oidium, Paecilomyces, papulospora, Penicillium, Periconia, perithecium, Peronospora, phaeohyphomycosis, Phoma, Pithomyces, RhizoMucor, Rhizopus, Rhodococcus, Rhodotorula, rusts, Saccharomyces, Scopulariopsis, Sepedonium, Serpula lacrymans, smuts, Spegazzinia, spore, Sporoschisma, Sporothrix, Sporotrichum, Stachybotrys, Stemphylium, Syncephalastrum, Thermononespore fusca DSM43793, Torula, Trichocladium, Trichoderma, Trichophyton, Trichothecium, tritirachium, Ulocladium, Verticillium, Wallemia, yeast, or any combination thereof.

### Embodiment 53

The superabsorbent polymer of any one of embodiments 45-52, wherein the superabsorbent polymer is degradable upon exposure to solar radiation.

### Embodiment 54

The superabsorbent polymer of embodiment 53, wherein the solar radiation comprises gamma rays, UV rays, visible light, infrared rays, x-rays, microwave radiation, radio waves, or any combination thereof.

### Embodiment 55

The superabsorbent polymer of any one of embodiments 45-52, wherein the superabsorbent polymer degrades upon contact with an effective amount of environmental moisture.

### Embodiment 56

The superabsorbent polymer of embodiment 55, wherein the environmental moisture comprises a body of water, soil moisture, waste moisture, or an atmospheric humidity.

### Embodiment 57

The superabsorbent polymer of embodiment 55 or 56, wherein environmental moisture comprises a relative humidity of 10% to 60% or a moisture content of from 10% to 65%, or greater.

### Embodiment 58

The superabsorbent polymer of any one of embodiments 19-57, wherein 75% or greater of the superabsorbent polymer is degraded at a temperature of 15° C. in 20 days when measured by ISO 20200:2024 or a comparable assay.

### Embodiment 59

The superabsorbent polymer of any one of embodiments 19-58, wherein 80% or greater of the superabsorbent polymer is degraded at a temperature of 15° C. in 20 days when measured by ISO 20200:2024 or a comparable assay.

### Embodiment 60

The superabsorbent polymer of any one of embodiments 19-59, wherein

85% or greater of the superabsorbent polymer is degraded at a temperature of 15° C. in 20 days when measured by ISO 20200:2024 or a comparable assay.

### Embodiment 61

The superabsorbent polymer of any one of embodiments 19-60, wherein 90% or greater of the superabsorbent polymer is degraded at a temperature of 15° C. in 20 days when measured by ISO 20200:2024 or a comparable assay.

### Embodiment 62

The superabsorbent polymer of any one of embodiments 19-61, wherein 95% or greater of the superabsorbent polymer is degraded at a temperature of 15° C. in 20 days when measured by ISO 20200:2024 or a comparable assay.

### Embodiment 63

The superabsorbent polymer of any one of embodiments 19-62, wherein 99% or greater of the superabsorbent polymer is degraded at a temperature of 25° C. in 20 days when measured by ISO 20200:2024 or a comparable assay.

### Embodiment 64

The superabsorbent polymer of any one of embodiments 19-63, wherein about 100% of the superabsorbent polymer is degraded at a temperature of 15° C. in 20 days when measured by ISO 20200:2024 or a comparable assay.

### Embodiment 65

A biodegradable poly(aspartic acid) hydrogel, comprising a superabsorbent biopolymer comprising a poly(aspartic acid) polymer crosslinked with 1,8-diaminooctane and/or cystamine; wherein the poly(aspartic acid) biopolymer comprises particles that are surface crosslinked with ethylene glycol diglycidyl ether (EGDGE), poly(ethylene glycol) diglycidyl ether (PEGDGE), or ethylene glycol and wherein the superabsorbent biopolymer has a whiteness index of L* in a range of from 97 to 100, a* in a range of from −1.4 to −1.2 and b* in a range of from 4.3 to 4.6.

### Embodiment 66

The biodegradable poly(aspartic acid) hydrogel of embodiment 65, wherein the superabsorbent biopolymer has a water absorption capacity of from 700 g/g to 1400 g/g.

### Embodiment 67

The biodegradable poly(aspartic acid) hydrogel of embodiments 65 or 66, comprising a free swell capacity (FSC) of from 30 g/g to 80 g/g under a 0.9% saline solution.

### Embodiment 68

The biodegradable poly(aspartic acid) hydrogel of any one of embodiments 65-67, comprising a centrifuge retention capacity (CRC) of from 30 g/g to 70 g/g.

### Embodiment 69

The biodegradable poly(aspartic acid) hydrogel of any one of embodiments 65-68, comprising an absorbance under load (AUL) of from 8 g/g to 20 g/g under a load of 0.7 psi.

### Embodiment 70

A method of preparing a superabsorbent polymer comprising: (a) polymerizing aspartic acid monomers using an acid catalyst to form a polysuccinimide polymer; (b) crosslinking the polysuccinimide polymer in an organic solvent using one or more crosslinking agents to form a crosslinked polysuccinimide polymer; (c) hydrolyzing the crosslinked polysuccinimide polymer using base to form a salt form of crosslinked poly(aspartic acid) polymer; and (d) surface crosslinking the poly(aspartic acid) polymer with a surface crosslinker comprising ethylene glycol diglycidyl ether (EGDGE) or poly(ethylene glycol) diglycidyl ether (PEGDGE), or ethylene glycol, wherein the superabsorbent polymer a whiteness index of L* in a range of from 97 to 100, a* in a range of from −1.4 to −1.2 and b* in a range of from 4.3 to 4.6.

### Embodiment 71

The method of embodiment 70, wherein the acid catalyst is a phosphoric acid (H₃PO₄) catalyst.

### Embodiment 72

The method of embodiment 70 or 71, wherein the base is sodium carbonate (Na₂CO₃) and the salt is a sodium salt.

### Embodiment 73

An article of manufacture comprising a superabsorbent polymer comprising a poly(aspartic acid) polymer crosslinked with 1,8-diaminooctane; wherein the superabsorbent polymer has a whiteness index of L* in a range of from 97 to 100, a* in a range of from −1.4 to −1.2 and b* in a range of from 4.3 to 4.6; a water absorption capacity in a range of from 700 g/g to 1400 g/g; and a free swell capacity (FSC) in a range of from 30 g/g to 80 g/g, and wherein the article of manufacture is a diaper, a sanitary pad, an incontinence product, a bandage, a wound dressing, a surgical pad, a pet pad, a bed pad, or a meat pad.

### Embodiment 74

The article of manufacture of embodiment 73, wherein article is substantially free of microplastics.

### Embodiment 75

The article of manufacture of embodiment 73 or 74, wherein the superabsorbent polymer comprises less than 5% monomers.

### Embodiment 76

A biodegradable poly(aspartic acid) hydrogel, comprising a superabsorbent polymer, comprising a poly(aspartic acid) polymer crosslinked with 1,8-diaminooctane; wherein the superabsorbent polymer has a water absorption capacity of from 10 g/g to 600 g/g; wherein the superabsorbent polymer has a saline absorption of from 10 g/g to 100 g/g; wherein the superabsorbent polymer has an absorption under load (AUL) of from 1 g/g to 100 g/g under a load of 0.7 psi; and wherein the biodegradable poly(aspartic acid) hydrogel is degraded upon hydrolysis at a temperature of 15° C. in 20 days when measured by ISO 20200:2024 or a comparable assay.

### Embodiment 77

A biodegradable poly(aspartic acid) hydrogel, comprising a superabsorbent polymer, comprising a poly(aspartic acid) polymer crosslinked with 1,8-diaminooctane and cystamine; wherein the superabsorbent polymer has a water absorption capacity of from 10 g/g to 600 g/g, and wherein the biodegradable poly(aspartic acid) hydrogel is degraded upon hydrolysis at a temperature of 15° C. in 20 days when measured by ISO 20200:2024 or a comparable assay.

### Embodiment 78

An article of manufacture comprising said biodegradable poly(aspartic acid) hydrogel of any one of embodiments 76 or 77.

### Embodiment 79

The article of manufacture of embodiment 78, wherein said article of manufacture is selected from the group consisting of: (a) devices for controlled release of water, nutrients or phytopharmaceuticals in agriculture, (b) absorbent products for dewatering mine tailings and sludge, and biomedical devices.

### Embodiment 80

A superabsorbent polymer, comprising a poly(aspartic acid) (PASP) polymer and one or more crosslinking agents, wherein the superabsorbent polymer has a water absorption capacity of at least 500 g/g, a saline absorption of from 10 g/g to 100 g/g, and an absorption under load (AUL) of at least 9 g/g under a load of 0.7 psi, wherein the superabsorbent polymer is degraded by at least 75% upon hydrolysis at a temperature of 15° C. or higher in 20 days when measured by ISO 20200:2024 or a comparable assay, wherein the one or more crosslinking agents, crosslinking the PASP polymer, are present at a concentration of at least 2.5 wt % of the superabsorbent polymer, and wherein the one or more crosslinking agents comprise 1,8-diaminooctane.

### Embodiment 81

A superabsorbent polymer, comprising a poly(aspartic acid) (PASP) polymer and one or more crosslinking agents, wherein the superabsorbent polymer has a water absorption capacity of at least 500 g/g, and wherein the superabsorbent polymer is degraded by at least 75% upon hydrolysis at a temperature of 15° C. in 20 days when measured by ISO 20200:2024 or a comparable assay, wherein the one or more crosslinking agents, crosslinking the PASP polymer, are present at a concentration of at least 2.5 wt % of the superabsorbent polymer, and wherein the one or more crosslinking agents comprise 1,8-diaminooctane and cystamine.

### Embodiment 82

The superabsorbent polymer of embodiment 80 or 81, comprising a free swell capacity (FSC) of from 20 g/g to 100 g/g under a 0.9% saline solution.

### Embodiment 83

The superabsorbent polymer of any one of embodiments 80-82, comprising a centrifuge retention capacity (CRC) of from 10 g/g to 50 g/g.

### Embodiment 84

The superabsorbent polymer of any one of embodiments 80-83, wherein the superabsorbent polymer comprises 50% or more biobased carbons.

### Embodiment 85

The superabsorbent polymer of any one of embodiments 80-84, wherein the 1,8-diaminooctane is present at a concentration of from 0.1 wt % to 15 wt % of the superabsorbent polymer.

### Embodiment 86

The superabsorbent polymer of any one of embodiments 81-85, wherein the cystamine is present at a concentration of from 0.1 wt % to 10 wt % of the superabsorbent polymer.

### Embodiment 87

A biodegradable poly(aspartic acid) hydrogel, comprising: a superabsorbent biopolymer comprising a poly(aspartic acid) polymer; one or more crosslinking agents; and one or more fluids, a charged species, a biological molecule, an organic compound, a metal, or combinations thereof, wherein the superabsorbent polymer has a water absorption capacity of at least 500 g/g, wherein the biodegradable poly(aspartic acid) hydrogel is degraded by at least 75% upon hydrolysis at a temperature of 15° C. in 20 days when measured by ISO 20200:2024 or a comparable assay, wherein the one or more crosslinking agents, crosslinking the poly(aspartic acid) polymer, are present at a concentration of at least 2.5 wt % of the superabsorbent polymer, and wherein the one or more crosslinking agents comprise 1,8-diaminooctane and cystamine.

### Embodiment 88

An article of manufacture comprising a superabsorbent polymer comprising a poly(aspartic acid) polymer; one or more crosslinking agents, wherein the superabsorbent polymer has a water absorption capacity of at least 500 g/g, wherein the superabsorbent polymer is degraded by at least 75% upon hydrolysis at a temperature of 15° C. in 20 days when measured by ISO 20200:2024 or a comparable assay, wherein the one or more crosslinking agents, crosslinking the poly(aspartic acid) polymer, are present at a concentration of at least 2.5 wt % of the superabsorbent polymer, wherein the one or more crosslinking agents comprise 1,8-diaminooctane and cystamine, and wherein the article of manufacture is a diaper, a sanitary pad, an incontinence product, a bandage, a wound dressing, a surgical pad, a pet pad, a bed pad, or a meat pad.

### Embodiment 89

A biodegradable poly(aspartic acid) hydrogel, comprising: a superabsorbent biopolymer comprising a poly(aspartic acid) polymer and one or more crosslinking agents, wherein the superabsorbent polymer has a water absorption capacity of at least 500 g/g, wherein the superabsorbent polymer has a saline absorption of from 10 g/g to 100 g/g, wherein the superabsorbent polymer has an absorption under load (AUL) of from 1 g/g to 100 g/g under a load of 0.7 psi, wherein the biodegradable poly(aspartic acid) hydrogel is degraded by at least 75% upon hydrolysis at a temperature of 15° C. in 20 days when measured by ISO 20200:2024 or a comparable assay, wherein the one or more crosslinking agents, crosslinking the poly(aspartic acid) polymer, are present at a concentration of at least 2.5 wt % of the superabsorbent polymer, and wherein the one or more crosslinking agents comprises 1,8-diaminooctane.

### Embodiment 90

A biodegradable poly(aspartic acid) hydrogel, comprising: a superabsorbent biopolymer comprising a poly(aspartic acid) polymer and one or more crosslinking agents, wherein the superabsorbent polymer has a water absorption capacity of at least 500 g/g, wherein the biodegradable poly(aspartic acid) hydrogel is degraded by at least 75% upon hydrolysis at a temperature of 15° C. in 20 days when measured by ISO 20200:2024 or a comparable assay, wherein the one or more crosslinking agents, crosslinking the poly(aspartic acid) polymer, are present at a concentration of at least 2.5 wt % of the superabsorbent polymer, and wherein the one or more crosslinking agents comprises 1,8-diaminooctane and cystamine.

What is claimed is:

1. A crosslinked polymer composition, comprising a poly(aspartic acid) (PASP) polymer, and two or more crosslinking agents, wherein the crosslinked polymer composition has:
a water absorption capacity of at least 500 g/g;
an absorption under load (AUL) of at least 16 g/g under a load of 0.7 psi;
a centrifuge retention capacity (CRC) of at least 30 g/g; and
a free swell capacity (FSC) of at least 40 g/g under a 0.9% saline solution,
wherein the two or more crosslinking agents comprises at least one diamine crosslinker that is present in an amount of from 1 wt % to 5 wt % of a total crosslinked polymer composition, and
wherein the crosslinked polymer composition comprises a crosslinked PASP polymer in a salt form.

2. The crosslinked polymer composition of claim 1, wherein the at least one diamine crosslinker selected from the group consisting of:
1,8-diaminooctane, cystamine, ethylenediamine, 1,4-butanediamine, 1,5-pentanediamine, 1,6-hexamethylenediamine, 1, 10-diaminodecane, 1,12-diaminododecane, L-lysine, L-ornithine, diethylenetriamine, triethylenetetramine, 1,4-diaminocyclohexane, p-phenylenediamine, tris(2-aminoethyl)amine.

3. The crosslinked polymer composition of claim 1, wherein the two or more crosslinking agents comprises at least one epoxy crosslinker selected from the group consisting of: ethylene glycol diglycidyl ether (EGDGE); poly(ethylene glycol) diglycidyl ether (PEGDGE); glycerol polyglycidyl ether, sorbitol polyglycidyl ether, and any combination thereof.

4. The crosslinked polymer composition of claim 1, wherein the crosslinked polymer composition comprises a crosslinked PASP polymer in a sodium salt form.

5. The crosslinked polymer composition of claim 1, wherein the crosslinked polymer composition degrades upon contact with an effective amount of environmental moisture up to at least 70%, as determined by ISO 20200:2024 or a comparable assay.

6. A crosslinked polymer composition, comprising a poly(aspartic acid) (PASP) polymer and two or more crosslinking agents, wherein the crosslinked polymer composition has:
a water absorption capacity of at least 500 g/g; and
a whiteness index of L* in a range of from 97 to 100, a* in a range of from −1.4 to −1.2 and b* in a range of from 4.3 to 4.6, and wherein
the two or more crosslinking agents comprise at least one diamine crosslinker as an internal crosslinker.

7. The crosslinked polymer composition of claim 6, further comprising an absorption under load (AUL) of at least 16 g/g under a load of 0.7 psi.

8. The crosslinked polymer composition of claim 6, further comprising a centrifuge retention capacity (CRC) of at least 30 g/g.

9. The crosslinked polymer composition of claim 6, comprising a free swell capacity (FSC) of at least 40 g/g under a 0.9% saline solution.

10. The crosslinked polymer composition of claim 6, wherein the at least one diamine crosslinker is selected from the group consisting of:
1,8-diaminooctane, cystamine, ethylenediamine, 1,4-butanediamine, 1,5-pentanediamine, 1,6-hexamethylenediamine, 1, 10-diaminodecane, 1,12-diaminododecane, L-lysine, L-ornithine, diethylenetriamine, triethylenetetramine, 1,4-diaminocyclohexane, p-phenylenediamine, tris(2-aminoethyl)amine.

11. The crosslinked polymer composition of claim 6, wherein the at least one diamine crosslinker is present in an amount of at least 2.5 wt % of the crosslinked polymer composition.

12. The crosslinked polymer composition of claim 6, wherein the two of more crosslinking agents comprise at least one epoxy crosslinker as a surface crosslinker.

13. The crosslinked polymer composition of claim 12, wherein the at least one epoxy crosslinker is present in an amount of at least 1.5 wt % of the crosslinked polymer composition.

14. The crosslinked polymer composition of claim 6, wherein the two or more crosslinking agents comprise at least one crosslinker selected from the group consisting of: ethylene glycol diglycidyl ether (EGDGE); poly(ethylene glycol) diglycidyl ether (PEGDGE); glycerol polyglycidyl ether, sorbitol polyglycidyl ether, and any combination thereof.

15. The crosslinked polymer composition of claim 6, wherein the crosslinked polymer composition degrades upon contact with an effective amount of environmental moisture up to at least 70%, as determined by ISO 20200:2024 or a comparable assay.

16. The crosslinked polymer composition of claim 6, that is substantially free of microplastics, wherein the microplastics are present in an amount of at least less than 0.1 wt %, less than 0.01 wt %, or less than 0.001 wt % of a total weight of the crosslinked polymer composition in accordance with European Union (EU) regulations under Commission Regulation (EU) 2023/2055 (Annex XVII), Registration, Evaluation, Authorization and Restriction of Chemicals (REACH).

17. An article of manufacture comprising a crosslinked polymer composition, comprising a poly(aspartic acid) (PASP) polymer and two or more crosslinking agents, wherein:
the crosslinked polymer composition has a water absorption capacity of at least 500 g/g;
a whiteness index of L* in a range of from 97 to 100, a* in a range of from −1.4 to −1.2 and b* in a range of from 4.3 to 4.6; and
the two or more crosslinking agents comprises at least one diamine crosslinker as an internal crosslinker, wherein the article of manufacture is selected from the group consisting of:
(a) a biomedical product; or
(b) a hygiene product.

18. The article of manufacture of claim 17, wherein:
the biomedical product is selected from the group consisting of: a wound dressing, a drug delivery hydrogel, a tissue engineering scaffold, a surgical pad, a drug delivery system, a tissue engineering product, and a surgical hygiene product.

19. The article of manufacture of claim 17, wherein:
the hygiene product is selected from the group consisting of: a disposable diaper, an adult incontinence pad, and a sanitary napkin.

20. An article of manufacture comprising a crosslinked polymer composition comprising poly(aspartic acid) (PASP) polymer and two or more crosslinking agents, wherein the crosslinked polymer composition has a water absorption capacity of at least 500 g/g; an absorption under load (AUL) of at least 16 g/g under a load of 0.7 psi; a centrifuge retention capacity (CRC) of at least 30 g/g; and a free swell capacity (FSC) of at least 40 g/g under a 0.9% saline solution, wherein the two or more crosslinking agents comprises at least one diamine crosslinker that is present in an amount of at least 2.5 wt %, and wherein the article of manufacture is selected from the group consisting of:
(a) an agricultural product;
(b) a biomedical product;
(c) a mining and/or mineral processing product;
(d) a hygiene product; or
a packaging product.
